SEQUENCE LISTING (13 sequences)

SEQ ID NO: 1 - DNA, 2358 bp, Mus musculus
ggatcctttcatgtttaacaatatcaaccctaacccaaggggaacagcctgcctgacagtggctttgccacccatgaatacttcctagtctagtccgtttgtgaaactcagcccatcccaacacttctgcaagccccatcctctacaaggtgctcattgggaatttcctggagcttctctttcaggatcagcctgattctagggcagcagttctcaacctgggggcctcgacccctttgggggaatcaaacgaccctttacaggggtcacatatcatctatcctatatgtcaggtatttacattacgattcgtaacagtagcaaaattacaggtatgaaatagcaatgaaataattttatgattgaaggtcaccacaacatgaggccgccacactgttctagagaaaaatcacctgggtggggaaaggtttgggaaagcctttctgtccattcttcattcttcaaagtgatgtgttcacagaaagcctttcagctgttctgctggggctcttagtaagtctgagtaggaactgtatgtaccaggtctgcttcttatgggtggagccaagacgcatcgtgggtggagcgaagacgcaacctcaccttctagctctgcatccatagcaagtagcctaatgtttctgtgtctaggtgtcatctctgtgaatcgagatccttggccttgcttgaattagggaggcacaaaatactcagagattcaagactgctcagcagcccagagtccttcctcaaaggaaaggtctcaactctcagccccccttagctctgagtcaggcctggaacaaacggccacaggaatgagaaaagctgccatagctgcttgtcacttcaagaggtcaaagaaaatagtgttaaccatgaaaacgagaagaccaacagttatccattgatagcgtctcaggacagataggacagagagaacactaggagaggggaacccacgaaggacaaggtattagtgtgttggttttcagggcaatgtcttgtactgaagattctagaaacacaatttgctggttgaacagctgaagtggggtgggggttcttaccccatgttcatggaagggtgagtgaggagagacagatatatgatggccagcataacaaacatacacaacaccctaattaacacttccctcttctactgacacccccttcactctcctctttcataaaaaataaaaaaagtattttatgtggctcttacgatagaatctttcctcgaactataaaaagatctaaatatttatatttttcacattttaatatcttagcgatgacaagccagaaacaagtattttttgcctctctcaacagcaaagcttggggcctttttgtttccgtgttaggaatagaacacgagagccccgtgtatctaggcagatgctctatcattagcccatgagtctccagcctcagacgcacatttttctcgggctctcttaagcttttcccacagcattgggaaactttactgacagcatccaagttgtgcttctgctaagaactggactcacatctctctgtgcatcacttcggcccgttttggggtagatcctctgattagccttcagatttagaacacggtgagcctgtggtgcactaattatggccagtgacaccatagagtcaaagtgcattactgaatgctttcaatttctcctaatgctggtacgatggcatgtcacagggccattttagctgcagacatcactccagagaattccaaacagatagagacaagtggcacccagacccatctccttcccctcgggctgattatccccagaaataggatgtcccaaagcaacacttcccagccaactggagtgctgataagtccagttatcagaaagatatggctgtaagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggccagcttgtcatctccctcttgggcttcccagacactaagtctggaatgaaaattcacctgcctctgaattggccactggtgggggcaggggtgtgacttggcttcccaggctggaagattatctcacccagccctagctatataacgggctggtgtggaggggctccacagggccagttccaggggttcatccacaagagagaaaaacatagactcgaggtctagggagcttgcatgcctgcaggtcggaggccaccatgg

SEQ ID NO: 2 - DNA, 2074 bp, Homo sapiens
ctgcagcaagttacttaatgttttttgcctcagcatcctctctgtaaaatgagagcattagtcttgctccaacttcgagggcatggacagctctgggatttcatatccaagacccttaaacatcccacagtccttcccccaaacacttctcctcctaatacctccctcagtttgggtcaggcctggaacaaaaaggcatacgaaatggtagaaaaagtgtccatgactacttctgacttagatgaagagaccaatgaaaatagtaatgactctgtttgcttcagcaggacatatactaaaataggagctatacaaagaagattagcatggactctgtgcaagaatgacacacaaatttgtgaaacattccatatattaaaaataaataaataataaagagaaaaggaaaaaattaaaaagaaaatagtgatagctgtgtccatctcaaagaaaagcccaggagatttcctttatttaccccctttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctgtcttttactsakaraaacccagtttttcctgaaactataaaaatacccccagtatgtttacataatttacacctcaaagattagaaaccagaaatagagaccttttcaacccttccggaagcaaagtgcattatccctccagccacgtgtctcaaatcttgatgcatcagaatcatctgggtgctttkaaattcaagatgattcctacgagttaccataaatcaactcagaattccctggagtggggccagggatctgtatttctgacaagctcccacaggtgattcctttccccacagcatttgagaacttcagctcaatgacctaatcagagtcctgccattgctaatatctggtctcatttttbtcatatatatatatagtatttgtggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatgtcttataatgatggtaaggtggcatgtcatggggcctatttagcccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacatacaagactccttcagccaac

SEQ ID NO: 3 - DNA, 2740 bp, Homo sapiens
gtgaacttttatgggaaggatgcttctgaaaaacaaatgacagaaaactctccgccaggggaatttttttctcaattttgatgaataagaacgatttgaaaatacaatggttgttgtttttatctttttagagagctaaaggtgcctagaatctcttttcaaaaagcagattctctcatgttttttttctttatttgttgtcatattctttttacatcttctgaccacttatcctcaagttgtacctctcatgttttataatgacaagctggatcaacatgggaaaaggttgaactggcagtgatttcaccagccctgacatccttgcatccaccagcgtgctcctttaagttcagcccattccatcaactcatcttcaagtgtcatcctctgcaaagttttcttcaagacttcctggagcctctctatagaatcagctaggtttcaagggataattaaatgcctggagaaagaaaagggcttggtaagcctccctgcccactttcacttgcattctttgaggtgattgaaacagtaaggagccatttaatcagttttggttgcatcctgagtgggtctaggtgagacttgccctaggaaatcttttgggctcaatgattgtctgcttctgttggatggaatcaggactcttcaacctagcattcaccaactagctgtgcatctgcagcaagttacttaatgtttctttgcctcagcatcctctctgtaaaatgagagcattagtcttgctccaacttcgagggcatggacagctctgggatttcatatccaagacccttaaacatcccacagtccttcccccaaacacttctcctcctaatacctccctcagtttgggtcaggcctggaacaaaaaggcatacgaaatggtagaaaaagtgtccatgactacttctgacttagatgaagagaccaatgaaaatagtaatgactctgtttgcttcagcaggacatatactaaaataggagctatacaaagaagattagcatggactctgtgcaagaatgacacacaaatttgtgaaacattccatatattaaaaataaataaataataaagagaaaaggaaaaaattaaaaagaaaatagtgatagctgtgtccatctcaaagaaaagcccaggagatttcctttaattaacccccttttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctttcttttactgaaaaaaacccagtttttcctgaaactataaaaataccccagtatttttacataatttacacctcaaagattagaaaccagaaatagagacctttttcaacccttccggaagcaaagtgcattatccctccagccacgtgtctcaaatcttgatgcatcagaatcatctgggtgctttgaaattcaagatgattcctacgagttaccataaatcaactcagaattccctggagtggggcccagggatctgtatttctgacaagctcccacaggtgattcctttccccacagcatttgagaacttcagctcaatgacctaatcagagtcctgccattgctaataactggtctcatttttttcatatatatatatagtatttttggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

SEQ ID NO: 4 - DNA, 2146 bp, Homo sapiens
taggaaatcttttgggctcaatgattgtctgcttctgttggatggaatcaggactcttcaacctagcattcaccaactagctgtgcatctgcagcaagttacttaatgtttctttgcctcagcatcctctctgtaaaatgagagcattagtcttgctccaacttcgagggcatggacagctctgggatttcatatccaagacccttaaacatcccacagtccttcccccaaacacttctcctcctaatacctccctcagtttgggtcaggcctggaacaaaaaggcatacgaaatggtagaaaaagtgtccatgactacttctgacttagatgaagagaccaatgaaaatagtaatgactctgtttgcttcagcaggacatatactaaaataggagctatacaaagaagattagcatggactctgtgcaagaatgacacacaaatttgtgaaacattccatatattaaaaataaataaataataaagagaaaaggaaaaaattaaaaagaaaatagtgatagctgtgtccatctcaaagaaaagcccaggagatttcctttaattaacccccttttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctttcttttactgaaaaaaacccagtttttcctgaaactataaaaataccccagtatttttacataatttacacctcaaagattagaaaccagaaatagagacctttttcaacccttccggaagcaaagtgcattatccctccagccacgtgtctcaaatcttgatgcatcagaatcatctgggtgctttgaaattcaagatgattcctacgagttaccataaatcaactcagaattccctggagtggggcccagggatctgtatttctgacaagctcccacaggtgattcctttccccacagcatttgagaacttcagctcaatgacctaatcagagtcctgccattgctaataactggtctcatttttttcatatatatatatagtatttttggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

SEQ ID NO: 5 - DNA, 2050 bp, Homo sapiens
agttacttaatgtttctttgcctcagcatcctctctgtaaaatgagagcattagtcttgctccaacttcgagggcatggacagctctgggatttcatatccaagacccttaaacatcccacagtccttcccccaaacacttctcctcctaatacctccctcagtttgggtcaggcctggaacaaaaaggcatacgaaatggtagaaaaagtgtccatgactacttctgacttagatgaagagaccaatgaaaatagtaatgactctgtttgcttcagcaggacatatactaaaataggagctatacaaagaagattagcatggactctgtgcaagaatgacacacaaatttgtgaaacattccatatattaaaaataaataaataataaagagaaaaggaaaaaattaaaaagaaaatagtgatagctgtgtccatctcaaagaaaagcccaggagatttcctttaattaacccccttttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctttcttttactgaaaaaaacccagtttttcctgaaactataaaaataccccagtatttttacataatttacacctcaaagattagaaaccagaaatagagacctttttcaacccttccggaagcaaagtgcattatccctccagccacgtgtctcaaatcttgatgcatcagaatcatctgggtgctttgaaattcaagatgattcctacgagttaccataaatcaactcagaattccctggagtggggcccagggatctgtatttctgacaagctcccacaggtgattcctttccccacagcatttgagaacttcagctcaatgacctaatcagagtcctgccattgctaataactggtctcatttttttcatatatatatatagtatttttggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

SEQ ID NO: 6 - DNA, 1582 bp, Homo sapiens
taacccccttttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctttcttttactgaaaaaaacccagtttttcctgaaactataaaaataccccagtatttttacataatttacacctcaaagattagaaaccagaaatagagacctttttcaacccttccggaagcaaagtgcattatccctccagccacgtgtctcaaatcttgatgcatcagaatcatctgggtgctttgaaattcaagatgattcctacgagttaccataaatcaactcagaattccctggagtggggcccagggatctgtatttctgacaagctcccacaggtgattcctttccccacagcatttgagaacttcagctcaatgacctaatcagagtcctgccattgctaataactggtctcatttttttcatatatatatatagtatttttggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

SEQ ID NO: 7 - DNA, 811 bp, Homo sapiens
acaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

SEQ ID NO: 8 - DNA, 739 bp, Homo sapiens
agagcccgctgccgccggagccgagccgacccgccccgccgacggtgagtcagcgcccggccctccgcgttcactcctcgcctggtccgcgggccgcgccggacgccagccccgcgccgccacctggccagcccggcccgcattcagccaaggccccagctcctgccgctctgcgactgcctttttttttttttttttaaagcccacactttttgatttggttctaacttgttttgtcctgggcgttggtcctcgcaggacctcgcaggggctctaagaaggggaattttgtggctccccaaggggcttttgggtccctactctcgtgcgctttcccctccatctggggcacaggcatggcgatatggacagggctggagatcgagttcccagttcgtgaaaaggaagaaagttaaagggctggggaggactaaggggctgggtttcttgggtccctccttgcacctggcaccctagctggaactcctggccagggagcctgggtggattcctctgcccttctctgtccccagtctcctccgcggcttcttccctcccttttatgattcgaggggaagggaggtggcaggagtgttccccgcccaaccccctgtccagtccccacaacccccttctgctctgtcctgtcctctgggtgcggagaaggccagctgcacaggcagctaagcgtggtccgccctcccctcctcaacctgcagaaccccctgaagctgtgcca

SEQ ID NO: 9 - DNA, 222 bp, Homo sapiens
agagcccgctgccgccggagccgagccgacccgccccgccgacggtgagtcagcgcccggccctccgcgttcactcctcgcctggtccgcgggccgcgccggacgccagccccgcgccggctagcctgtcctctgggtgcggagaaggccagctgcacaggcagctaagcgtggtccgccctcccctcctcaacctgcagaaccccctgaagctgtgccacc

SEQ ID NO: 10 - DNA, 219 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaacc

SEQ ID NO: 11 - DNA, 30 bp, Artificial Sequence
  PCR Primer
ggcgatttaaataatgtagtcttatgcaat

SEQ ID NO: 12 - DNA, 31 bp, Artificial Sequence
  PCR Primer
ggggtctagaaggtgcacaccaatgtggtga

SEQ ID NO: 13 - DNA, 3058 bp, Homo sapiens
ttgctttaggtgctacttctctgcttctcactttctccagctataaccatggtcctaattctagtcacatgtcatttcacccatggaaatgcataaatcctgaggggagtgggaaaaggctcatggggtgacactggagaagctcagggatgcttcctttactctttctggttggagatgggtgatgccaagttgctttatgattgtagaaccaactaggacctttattgttttaattcatcttagtaaggatagattatgtccagattgaggctatgataaagccaaatacacaaatataagaatttacaccactgggtgaacttttatgggaaggatgcttctgaaaaacaaatgacagaaaactctccgccaggggaatttttttctcaattttgatgaataagaacgatttgaaaatacaatggttgttgtttttatctttttagagagctaaaggtgcctagaatctcttttcaaaaagcagattctctcatgttttttttctttatttgttgtcatattctttttacatcttctgaccacttatcctcaagttgtacctctcatgttttataatgacaagctggatcaacatgggaaaaggttgaactggcagtgatttcaccagccctgacatccttgcatccaccagcgtgctcctttaagttcagcccattccatcaactcatcttcaagtgtcatcctctgcaaagttttcttcaagacttcctggagcctctctatagaatcagctaggtttcaagggataattaaatgcctggagaaagaaaagggcttggtaagcctccctgcccactttcacttgcattctttgaggtgattgaaacagtaaggagccatttaatcagttttggttgcatcctgagtgggtctaggtgagacttgccctaggaaatcttttgggctcaatgattgtctgcttctgttggatggaatcaggactcttcaacctagcattcaccaactagctgtgcatctgcagcaagttacttaatgtttctttgcctcagcatcctctctgtaaaatgagagcattagtcttgctccaacttcgagggcatggacagctctgggatttcatatccaagacccttaaacatcccacagtccttcccccaaacacttctcctcctaatacctccctcagtttgggtcaggcctggaacaaaaaggcatacgaaatggtagaaaaagtgtccatgactacttctgacttagatgaagagaccaatgaaaatagtaatgactctgtttgcttcagcaggacatatactaaaataggagctatacaaagaagattagcatggactctgtgcaagaatgacacacaaatttgtgaaacattccatatattaaaaataaataaataataaagagaaaaggaaaaaattaaaaagaaaatagtgatagctgtgtccatctcaaagaaaagcccaggagatttcctttaattaacccccttttaagatagaatattaggagaccggaacatatgatacaggaggtactgggagggtccctctttgtcaatgttttgtcttggggtggggagtcgatgtcttctcaaagtttcagaaacaccatccactgactgagcattcaaggggcaagaggagaatggcagccacatttgttgattgggtgagtttggggagaaatagacacacaaaggtcaaacataacttcctaattaacacttccctccattcacaattcccttctcccattcttctctcctttcttttactgaaaaaaacccagtttttcctgaaactataaaaataccccagtatttttacataatttacacctcaaagattagaaaccagaaatagagacctttttcaacccttccggaagcaaagtgcattatccctccagccacgtgtctcaaatcttgatgcatcagaatcatctgggtgctttgaaattcaagatgattcctacgagttaccataaatcaactcagaattccctggagtggggcccagggatctgtatttctgacaagctcccacaggtgattcctttccccacagcatttgagaacttcagctcaatgacctaatcagagtcctgccattgctaataactggtctcatttttttcatatatatatatagtatttttggtagagatgggattttgccatgttgcccaggctagtattgaactcctaagctaagcaatcttcctgtctctgcctcccaaaatgttgggattacaggtgtaagccactgcacccggctgatagctggtttcatttactctatttcttgaccactctgatccattttgaagtaaaaatgctccaattattatgctgttttagaacacggtaagcatgtcatgtgctaatggccagtgacatcataaaagaaaagtgcattactgaatgctttcaatttcttataatgatggtaaggtggcatgtcatggggcctatttagccccagacatcactccaaagaattccaaacagatatagacaagtgcctttagggcccagatcccttcccctcaggctgtttacccagggaataggatgtcctgggacaagtttcccctaagtgaagtgttgataagtctgcttatcagaaagatattactgggggtgtgatatgtagggcatctacattttcttgataggtagtcatatgaaagctgacaaagaaaaaaagggcagtgatgtggtgcaatgtcaacagacagctgtcccctgactcttgacaaataggatgacttgcattgctgagcgatgtgatcaccaccaaaggaatggccctctcacatttcttcctgattcacatattcagcagggttagcttgtcctcccctccctcttcagcttcccagacactgagtctggaatgaaaattcacctgcctctgagttggctcctaatgggggcgggagtgttacttcggttcccaggttggaagattatctcacccggccccagctatataagctgaccggtgtggaggggcccagcagggccaactccagggattccttccacgacagaaaaacc

(19) United States
(12) Patent Application Publication
(10) Pub. No.: US 2005/0004058 A1
(43) Pub. Date: Jan. 6, 2005

(54) Sequences upstream of the carp gene, vectors containing them and uses thereof

(76) Inventors: Patrick Benoit, Paris (FR); Bertrand Schwartz, Jouy En Josas (FR); Didier Branellec, Lyon (FR); Kenneth R. Chien, La Jolla, CA (US); Ju Chen, San Diego, CA (US)

Correspondence Address: Finnegan, Henderson, Farabow,;Garrett & Dunner, L.L.P., 1300 I Street, N.W., Washington, DC, 20005-3315, US

(21) Appl. No.: 10/802,030

(22) Filed: Mar. 17, 2004

Related U.S. Application Data
(63) Continuation-in-part of application No. 10/005,337, filed on Dec. 7, 2001.
(60) Provisional application No. 60/251,582, filed on Dec. 7, 2000.

(51) Int. Cl. A61K 48/00; C12Q 1/68; C07H 21/04; C12N 9/64

(52) U.S. Cl. 514/44; 435/6; 435/320.1; 435/325; 435/226; 536/23.2; 435/69.1

(57) ABSTRACT

The invention relates to novel promoter sequences derived from a portion upstream of the coding sequence of the gene for the CARP protein (Cardiac Ankyrin Repeat Protein), and which are capable of controlling the level and the specificity of expression of a transgene in vivo in cardiac muscle cells. The invention thus describes novel compositions, constructs, vectors and their uses in vivo for the transfer and expression of a nucleic acid in vivo in cardiac muscle cells. The subject of the present invention is also the use of the promoter sequences for generating transgenic animals which constitute models for studying certain cardiac pathologies.

This application is a continuation-in-part of U.S. Patent Application No. 10/005,337, filed Dec. 7, 2001, which claims the benefit of U.S. Provisional Application No. 60/251,582, filed Dec. 7, 2000, both of which are incorporated herein in their entirety.

The present invention relates to the field of biology. It relates in particular to the field of the targeting of the expression of genes, and more particularly the design and the development of a novel system for the specific expression of transgenes. The subject of the invention is, in particular, novel promoter sequences capable of controlling the level and the specificity of expression of a transgene in vivo in cardiac muscle cells. The invention thus describes novel compositions, constructs and vectors that make it possible to control and to direct the expression of a nucleic acid in cardiac muscle cells. The applications stemming from the present invention are numerous, for example in the experimental, clinical, therapeutic and diagnostic fields, and more particularly for the treatment and/or prevention of certain cardiac pathologies.

The control of the level and of the targeting of the expression of transgenes is necessary for many applications. For example, in gene therapy the success of the therapy may require targeting of the protein synthesized from the transgene and thus make it possible to limit the spread of side effects. The construction of transgenic animals and the study of the effects of a gene are additional examples in which an appropriate control of the specificity of expression of a protein can be used and can provide improvements.

In this regard, many promoters have been tested for their capacity to direct a cardiospecific expression. They are in particular the promoters of the gene encoding the cardiac myosin light chain (MLC-2) in rats (Henderson S. A. et al., J Biol Chem, 264 (1989) 18142-8; Lee K. J. et al., J Biol Chem, 126 (1992) 15875-85), cardiac α-actin in mice (Biben C. et al., Dev Biol, 173 (1996) 200-12), atrial natriuretic factor (ANF) (Harris A. N. et al., J Mol Cell Cardiol, 29 (1997) 515-25), α- or β-myosin heavy chain (α- or β-MHC) (Colbert M. C. et al., J Clin Invest, 100 (1997) 1958-68), muscle creatine kinase (MCK) in rabbits (Vincent C. K. et al., Mol Cell Biol, 13 (1993) 567-74), or cardiac troponin T (U.S. Pat. No. 5,266,488).

While these promoters are known to confer a degree of tissue specificity, it is also known that their levels of activity remain well below those of so-called strong promoters, generally by a factor of between 10 and 100, such that a therapeutic use cannot really be envisaged.

By way of example, Franz W. M. et al., (Cardiovasc Res, 35 (1997) 560-6) and Griscelli F. et al., (C R Acad Sci III, 320 (1997) 103-12) have shown that the levels of activity of the sequences upstream of the genes encoding rat α-MHC and MLC-2 in adenoviral constructs remain substantially lower than those of the RSV (Rous sarcoma virus) promoter, by a factor of about 10.

The present application, therefore, relates to a novel promoter sequence derived from the region upstream of the CARP (Cardiac Ankyrin Repeat Protein) gene. This sequence is capable not only of directing a cardiospecific expression, but also exhibits a high level of expression in vivo, comparable to that of a strong promoter such as the CMV (cytomegalovirus) promoter.

The CARP protein, which constitutes one of the first markers for differentiation of cardiomyocytes acting downstream of the homeobox gene Nbx2.5 in the regulation of the expression of the MLC-2v gene, has been studied and the coding portion of its gene has been sequenced in mice (Zou Y. et al., Development, 24 (1997) 793-804), in rabbits (Aihara Y. et al., Biochim Biophys Acta, 28 (1999) 318-24), and in humans (Chu W. et al., J Biol Chem, 270 (1995) 10236-45).

Kuo H. et al. (Development, 126 (1999) 4223-34) have cloned a 10 kb fragment and sequenced a 2.5 kb fragment upstream of the coding sequence of the mouse CARP gene. Deletions from the 5′-end of the fragment were made and showed that a region of 213 bp of the promoter between nucleotides −166 and +47, relative to the transcription start position +1, was sufficient to confer cardiospecific expression in vitro, which suggested the presence, at the 5′-end, of an element for controlling the specificity of the promoter. Kuo et al. also generated transgenic mouse lines comprising a fragment of 2.5 kb upstream of the CARP gene, showing specific expression of a transgene in cardiac and skeletal muscle cells at an early stage of embryonic development, this expression then being inhibited during development.

Application WO 00/15821 describes a portion 5′ of the coding sequence of the mouse CARP gene, situated between nucleotides −2285 and +62, relative to the transcription start position +1. This sequence was evaluated in particular for its in vivo activity in adenoviral vectors. The levels of activity obtained remain very low, however, such that it was found to be necessary, in order to detect an activity in vivo, to isolate the promoter sequence between two inverted terminal repeats of an adeno-associated virus (AAV-ITR).

The Applicants focused on better characterizing the region upstream of the CARP gene protein-coding region. We were thus able to identify a novel sequence upstream of the CARP gene and demonstrate unexpected and advantageous properties of this novel sequence, in particular, a significant improvement in the level of activity in vivo.

The Applicants have discovered, surprisingly, that while this newly identified sequence conferred no significant expression in vitro, it was, on the contrary, possible to obtain very good levels of activity in vivo, equivalent to those of so-called strong promoters, while preserving a high selectivity for expression in cardiac tissue.

The subject of the present invention is therefore a polynucleotide comprising a portion upstream of the coding sequence of the gene for the CARP protein, or of a polynucleotide hybridizing under highly stringent conditions with said upstream sequence, the polynucleotide being capable of inducing specific expression in cardiac tissue of a transgene placed under its control.

The invention also relates to any polynucleotide of natural origin or which is obtained by chemical synthesis, exhibiting at least 93%, preferably at least 95%, identity with SEQ ID NO: 1. In a further embodiment of the invention, the polynucleotide exhibits at least 98% identity with SEQ ID NO: 1.

The term “polynucleotide of natural origin” is understood to mean a genomic DNA fragment obtained by cleaving cellular DNA with the aid of a restriction enzyme.

The term “polynucleotide obtained by chemical synthesis” is understood to mean a DNA fragment generated by automated or manual synthesis, for example, with the aid of a suitable automated apparatus.

For the present invention, the term “highly stringent conditions” is used in the sense given by Maniatis et al. 1982 (Molecular Cloning, A Laboratory Manual, Cold Spring Harbor CSH, N.Y., USA) or one of its more recent editions. By way of example, the hybridization conditions are such that three washes at 65° C. in the presence of 0.2×SSC, and 0.1% SDS are necessary in order to eliminate the nonhybridized fragments.

The “specific” character of transgene expression means that the activity of the promoter is significantly higher in cells of cardiac tissue. Although nonspecific expression can be observed in other cells, the corresponding level of activity remains very low (negligible) compared with that observed in cardiac cells, in general lower by a factor of at least 10.

The results presented in the examples show, in this regard, a difference in expression that may reach a factor of 1000, which reflects the high selectivity of the polynucleotides according to the invention for cardiac cells in vivo.

Moreover, the results presented in the examples below clearly show that the use of the polynucleotides of the invention offers a system for high levels of expression, above those for other promoters known to be specific for cardiac tissue, it being possible for the difference to exceed a factor of 100. These elements, therefore, illustrate the advantages and unexpected properties of the polynucleotide according to the invention, in terms of promoter strength and specificity, for the expression of nucleic acids of interest in the cardiac tissue.

In one embodiment, the polynucleotide according to the invention comprises a portion of the sequence between −2266 and +92 (SEQ ID NO: 1), relative to transcription start position +1 of the CARP gene.

A subject of the present invention is therefore the sequences hybridizing, under high stringency conditions, with the sequence SEQ ID NO: 1.

The present invention is nevertheless not restricted to the polynucleotides containing fragments upstream of the mouse gene but relates to any functional variant or any other sequence of any other species having the same properties, namely being capable of specifically inducing expression in vivo of a transgene in cardiac tissue.

Thus, persons skilled in the art will be able to refer to the sequence upstream of the human gene deposited in GenBank under the reference AF131884 (SEQ ID NO: 2). The present invention thus encompasses any sequence comprising fragments of the sequences upstream of the gene for the CARP protein, modified, for example, by deletion of certain structures and which preserve identical or similar functions to that of the sequence SEQ ID NO: 1.

In one embodiment of the invention, the polynucleotide has at least 80% identity with SEQ ID NO: 2. In another embodiment of the invention, the polynucleotide has at least 90% identity with SEQ ID NO: 2. In another embodiment of the invention, the polynucleotide has at least 95% identity with SEQ ID NO: 2.

In another embodiment, a polynucleotide according to the invention comprises a portion of the sequence between −2702 and +38 (SEQ ID NO: 3), relative to transcription start position +1 of the human CARP gene, or any functional variant or sequence capable of hybridizing with said sequence under stringent conditions of hybridization, or that has at least 90% or about 95% identity with SEQ ID NO: 3.

Other polynucleotides according to the invention comprise a portion of the sequence between −2108 and +38 (SEQ ID NO: 4), or between −2011 and +38 (SEQ ID NO: 5), relative to transcription start site position +1 of the human CARP gene, or any functional variant or sequence capable of hybridizing with said sequences under stringent conditions of hybridization, or that has at least 90% or about 95% identity with SEQ ID NOs: 4 or 5.

In another embodiment, the polynucleotide according to the invention comprises a portion of the sequence between −1543 and +38 (SEQ ID NO: 6), relative to transcription start site position +1 of the human CARP gene, or any functional variant or sequence capable of hybridizing with said sequence under stringent conditions of hybridization, or that has at least 90% or about 95% identity with SEQ ID NO: 6.

Another polynucleotide according to the invention comprises a portion of the sequence between −772 and +38 (SEQ ID NO: 7), relative to transcription start site position +1 of the human CARP gene, or any functional variant or sequence capable of hybridizing with said sequence under stringent conditions of hybridization, or that has at least 90% or about 95% identity with SEQ ID NO: 7.

Applicants have discovered that 5′ deletions of the CARP promoter retain activity and tissue specificity in vivo, thereby allowing a reduction in the total length of the vector without modifying the strength of the promoter in vivo. In contrast, 5′ deletions of the human CARP promoter result in a decrease of the transcriptional activity in vitro, with the exception of the sequence comprising −1543 to +38 (SEQ ID NO: 6), relative to transcription start site position +1, which provided maximal transcriptional activity in vitro.

The term “functional variant” is understood to mean any modified sequence preserving the properties of the polynucleotides as mentioned above. The modifications may comprise one or more additions, mutations, deletions and/or substitutions of nucleotides in the sequence considered. These modifications may be introduced by conventional molecular biology methods, such as, for example, site-directed mutagenesis, or by artificial synthesis of the sequence. The variants obtained are then tested for their capacity to mediate specific expression in cardiac muscle cells when compared to a polynucleotide having the sequence of SEQ ID NO: 1.

Another subject of the invention is an expression cassette comprising a polynucleotide as defined above operably linked to a transgene such that the expression of the latter is specifically directed in cardiac muscle.

An expression cassette according to the invention may also comprise a signal for the termination of transcription and a late polyadenylation signal at the 3′-end of the nucleotide sequence of the transgene.

Polyadenylation (polyA) signals are well known to one of ordinary skill in the art. In one embodiment of the invention, polyA signals are late polyA signals from simian virus 40 (SV40) or human growth hormone (hGH polyA; e.g., SEQ ID NO: 10).

In one embodiment of the invention, the polynucleotide sequences of the invention are combined with the 5′-untranslated region (UTR) of the human cardiac α-actin promoter. In a particular embodiment, the 5′-UTR sequence used in the constructs corresponds to the sequence between +1 and +739 (SEQ ID NO: 8) minus the sequence between +119 to +645 (SEQ ID NO: 9) of the human cardiac α-actin promoter.

In one embodiment, the transgene comprises a nucleic acid encoding a protein or an RNA of therapeutic interest, which may, for example, be involved in cardiac pathologies such as cardiac insufficiency, cardiac hypertrophy, hypoxia, ischemia, or in cardiac transplant rejection.

As proteins of therapeutic interest, there may be mentioned, inter alia:
proteins inducing angiogenesis, such as, for example, members of the vascular endothelial growth factor (VEGF) family, members of the fibroblast growth factor (FGF) family and, more particularly, FGF1, FGF2, FGF4, FGF5, angiogenin, epidermal growth factor (EGF), transforming growth factor (TGF) α, TGFβ, tumor necrosis factor (TNF), scatter factor/hepatocyte growth factor (HGF), members of the angiopoietin family, cytokines and interleukins including IL-1, IL-2, IL-8, angiotensin-2, tissue plasminogen activator (TPA), urokinase (uPA), and molecules involved in the synthesis of active lipids (e.g., prostaglandins, Cox-1);
proteins involved in the control of cardiac contractility, such as phospholamban, phospholamban inhibitors, sarco-endoplasmic reticulum Ca(2+) ATPase-2a (SERCA-2a), β2-adrenergic receptor, and dystrophin or minidystrophin (FR91 11947);
proteins with cryoprotective activity, which block apoptosis, such as proteins which are members of the bcl family, and protein kinases such as AKT/PKB;
transcription factors, including, for example, natural or chimeric nuclear receptors, comprising a DNA-binding domain, a ligand-binding domain, and a transcription activating or inhibiting domain, such as, for example, the fusion proteins tetR-NLS-VP16, the fusion proteins derived from estrogen receptors, the fusion proteins derived from steroid hormone receptors, the fusion proteins derived from progesterone receptors, and the proteins of the CID (Chemical Inducer of Dimerization) system described by Rivera et al., (Rivera et al., Nature Medicine, 2 (1996) 1028-1032). There may be mentioned, in particular, as chimeric nuclear receptors, the nuclear receptors PPAR (Peroxisome Proliferator Activated Receptor) and PPAR2, as described in Applications WO 96/23884 and FR 99 07957, and by Frohnert et al., (J Biol Chem 274 (1999) 3970-3977), and by Mukherjee et al., (J Biol Chem 272 (1997) 8071-8076), either in its native form, without modification of the primary structure, or a modified PPAR2 comprising one or more ligand-binding sites or E/F domains (Schoonjans et al. Biochim. Biophys. Acta. 1302 (1996) 93-109), such as PPAR22 having the sequence of SEQ ID NO: 3;
immunosuppressors such as, for example, interleukins 2 and 10 that make it possible to completely or partially inhibit an immune signaling pathway and, thus, to extend the duration of cardiac transplants;
proteins involved as agents for reducing hypoxia, such as NOS (nitric oxide synthetase), B-cell leukemia/lymphoma 2 (bcl-2), superoxide dismutase (SOD) and catalase.

As RNAs of therapeutic interest, there may be mentioned, for example, antisense RNAs, which are useful for controlling the expression of genes or the transcription of cellular mRNAs, thus blocking translation into a protein according to the technique described in Patent EP 140 308, as well as ribozymes that are capable of selectively destroying target RNAs as described in EP 321 201.

It is understood that the present invention is not limited to these specific examples of proteins or RNAs, but that it can be used by persons skilled in the art for the expression of any nucleic acid in cardiac cells by simple, customary, experimental operations.

The subject of the present invention is additionally a vector containing the polynucleotide or the expression cassette according to the invention. Such a vector may contain any other DNA sequence necessary or useful for the expression of the transgene in target tissues and, in particular, may contain a replication origin that is effective in the cardiac cells.

The vector of the invention may be of various natures and/or origins, for example, plasmid, cosmid, episomal, chromosomal, viral, or phage. In one embodiment, the vector is either a plasmid or a recombinant virus.

By way of illustration of the plasmids according to the invention comprising a polynucleotide or an expression cassette, there may be mentioned, for example, the plasmids pXL3634, pXL3728, pXL3759, pXL4254, pXL4253, pXL4269, pXL4237, pXL4255, pXL4330, and pXL4331, which are described below.

According to one embodiment, the vectors according to the invention are of the plasmid type. As plasmid vectors, there may be mentioned, inter alia, any cloning and/or expression plasmids known to a person skilled in the art, which generally comprise an origin of replication. There may also be mentioned new-generation plasmids carrying replication origins and/or markers that have been refined, as described, for example, in Application WO 96/26270.

According to another embodiment, the plasmid vector is a miniplasmid and comprises an origin of replication whose functionality in the host cell requires the presence of at least one protein that is specific and foreign to the cell. Such vectors are described, for example, in Application WO 97/10343.

According to another embodiment, the vectors according to the present invention are viral vectors. Among the latter, there may be mentioned, inter alia, recombinant adenoviruses, recombinant adeno-associated viruses, recombinant retroviruses, lentiviruses, herpesviruses, and vaccinia viruses, whose preparation may be carried out according to methods known to persons skilled in the art. Chimeric viral vectors may be used, such as the adenovirus-retrovirus chimeric vectors that are described, inter alia, in Application WO 95/22617, as well as the episome/adenovirus vectors that are described by Leblois et al. (Mol Ther(2000) 1(4), 314-322) and in Application WO 97/47757.

When adenoviruses are used according to this embodiment, these are preferably vectors derived from defective adenoviruses, that is to say that they are incapable of autonomously replicating in the target cell. The construction of these defective viruses as well as their infectious properties have been widely described in the literature (see e.g., S. Baeck and K. L. March, Circul. Research, 82, (1998) 295-305; T. Shenk, B. N. Fields, D. M. Knipe, P. M. Howley et al. (1996), Adenoviridae: Viruses and Replication (in virology) 211-2148, EDS—Raven Publishers, Philadelphia; Yeh, P. et al. FASEB 11 (1997) 615-623).

Various adenovirus serotypes, whose structure and properties vary somewhat, have been characterized. Among these serotypes, use may be made in the context of the present invention, for example, of the type 2 or type 5 human adenoviruses (Ad 2 or Ad 5), or adenoviruses of animal origin, such as those described in Application FR 93 05954, or adenoviruses of mixed origin. Among the adenoviruses of animal origin that may be used in the context of the present invention, there may be mentioned the adenoviruses of canine, bovine, murine (Beard et al., Virology 75 (1990) 81), ovine, porcine, avian or simian origin. In one embodiment, the adenovirus of animal origin is a canine adenovirus, which may, for example, be a CAV2 adenovirus (Manhattan or A26/61 strain) as described in Application WO 94/26914.

The defective adenoviruses of the invention generally comprise an inverted terminal repeat (ITR) at each end, a sequence allowing encapsidation (Psi), the E1 gene, with at least one of the genes E2, E4 and L1-L5 having been inactivated by any technique known to persons skilled in the art (Levero et al., Gene, 101 (1991) 195, EP 185 573; Graham, EMBO J. 3 (1984) 2917).

In one embodiment, the recombinant adenovirus used in the invention comprises a deletion in the E1 region of its genome. This deletion may, for example, comprise a deletion of the E1a and E1b regions. By way of a specific example, there may be mentioned deletions affecting nucleotides 454-3328, 382-3446 or 357-4020 (with reference to the genome of Ad5).

According to another embodiment, the recombinant adenovirus used in the invention comprises, in addition to a deletion in the E1 region, a deletion in the E4 region of its genome. More particularly, the deletion in the E4 region affects all the open reading frames. There may be mentioned, by way of a specific example, deletion of nucleotides 33466-35535 or 33093-35535, again with reference to the genome of Ad5. Other types of deletions in the E4 region are described in applications WO 95/02697 and WO 96/22378, which are incorporated by reference into the present application.

Adeno-associated viruses (AAV) are relatively small-sized DNA viruses, which integrate into the genome of infected cells in a stable and site-specific manner. AAV can infect a broad spectrum of cells without having any effect on cell growth, morphology or differentiation. Moreover, AAV does not appear to be involved in pathologies in humans. The AAV genome has been cloned, sequenced and characterized. It comprises about 4700 bases and contains, at each end, an inverted terminal repeat (ITR) of about 145 bases, which serves as an origin of replication for the virus. The remainder of the genome is divided into 2 essential regions carrying the encapsidation functions: the left portion of the genome, which contains the rep gene involved in viral replication and in the expression of the viral genes, and the right portion of the genome, which contains the cap gene encoding the virus capsid proteins.

The use of AAV-derived vectors for the transfer of genes in vitro and in vivo has been described in the literature (see in particular WO 91/18088; WO 93/09239; U.S. Pat. No. 4,797,368, U.S. Pat. No. 5,139,941, EP 488528). These patent applications describe various AAV-derived constructs in which the rep and/or cap genes have been deleted and replaced with a gene of interest, and the use of these constructs for transferring in vitro (into cells in culture) or in vivo (into cells in an organism) the gene of interest. The defective recombinant AAVs according to the invention may be prepared by co-transfection, into a cell line infected with a human helper virus (for example, an adenovirus), of a plasmid containing the nucleic sequences of the invention bordered by two MV inverted terminal repeats (ITR) and of a plasmid carrying the MV encapsidation genes (rep and cap genes). The recombinant AAVs produced are then purified by conventional techniques.

Lentiviruses also may be used in the invention. They allow the transfer and the efficient and stable integration of a gene of interest into quiescent cells. There may be mentioned, for example, HTLV-1 and animal lentiviruses, such as FIV (feline infections virus), EIAV (equine infectious anemia virus; WO 98/51810), BIV (bovine immunodeficiency virus), SIV (simian immunodeficiency virus), CAEV (caprine arthritisencephalitis virus) (WO 98/39463; Naldini et al. Science 272 (1996) 263-267; Schnele et al. Hum Gen Ther 11 (2000) 439-447), or a lentivirus related to the one that causes AIDS, HIV-2, which is not highly pathogenic in humans (Kundra et al., Hum Gen Ther 9 (1998) 1371-1380).

The expression cassette may be inserted at various sites of the recombinant genome. It may be inserted in the E1, E3, or E4 region, as a replacement for suppressed or surplus sequences. It may also be inserted at any other site, outside of the sequences necessary in cis for the production of the viruses (ITR sequences and the encapsidation sequence).

It will be noted, however, that the introduction of the sequences according to the present invention into the vectors described above is not essential. That is, cardiac cells may be directly transfected with DNA comprising these sequences.

The nucleic sequences according to the present invention may be introduced after covalent coupling of the nucleic acid to compounds that promote their penetration into cells or their transport to the nucleus, the resulting conjugates being, optionally, encapsidated into polymeric microparticles, as in International Application WO 94/27238.

According to another embodiment, the nucleic sequences of the invention may be included in a transfection system comprising polypeptides promoting their penetration into cells, as in International Application WO 95/10534.

The polynucleotides, cassettes and vectors of the invention may be administered in situ by any means known to persons skilled in the art, for example, by coronary infusion (Barr et al., Gene Ther, 1, (1994) 51-58), by intracardiac injection, by epicardiac injection, that is to say through the ventricular wall (Guzman et al., Cir Res, 73 (1993) 1202-1207), by intrapericardiac injection (Fromes et al., Gene Ther, 6 (1999) 683-688), or by retrofusion of the coronary veins (Boeckstegers et al., Circulation, 100 (Suppl I) (1999), I-815).

The polynucleotides, cassettes, or vectors according to the invention may be administered as part of a composition containing them, for example, with the aid of a chemical or biochemical transfer agent facilitating their transfection into cardiac cells. The phrase “chemical or biochemical transfer agent” is understood to mean any compound facilitating the penetration of a nucleic acid into a cell. This may include cationic agents such as cationic lipids, peptides, polymers (polyethylenimine, polylysine), nanoparticles, and non-cationic agents, such as non-cationic liposomes, non-cationic nanoparticles, or polymers. Such agents are well known to persons skilled in the art and are, for example, described in applications WO 95/18863, WO 97/18185 and WO 98/15639.

The present invention, in addition, relates to medicaments containing such polynucleotides, expression cassettes or vectors, as well as to pharmaceutical compositions containing them in a pharmaceutically-effective quantity, as well as pharmaceutically-compatible excipients.

Such polynucleotides, expression cassettes, or vectors may be used for the manufacture of medicaments for delivery to cardiac tissue, which may express a gene encoding a protein of interest for the treatment of cardiac diseases, for example, for the treatment and/or prevention of cardiac insufficiency, hypoxia, cardiac hypertrophy, myocarditis, cardiac ischemia, or for preventing rejection after cardiac transplant.

Such a medicament may, for example, comprise a cassette or vector according to the invention that is capable of expressing the functional form of an impaired gene according to the cardiac pathology that it is desired to treat.

Preferably, the pharmaceutical composition contains pharmaceutically-acceptable vehicles for an injectable formulation, for example, for intracardiac injection. This may include, for example, isotonic, sterile saline solutions (monosodium or disodium phosphate, sodium, potassium, calcium or magnesium chloride, and the like, or mixtures of such salts), or dry, for example, freeze-dried, compositions, which, upon addition of sterilized water or of physiological saline, as appropriate, allow the preparation of injectable solutions. Other excipients may be used, such as, for example, a hydrogel. This hydrogel may be prepared using any biocompatible and non-cytotoxic (homo or hetero) polymer. Such polymers have been described, for example, in application WO 93/08845. Some of them, such as those obtained from ethylene and/or propylene oxide, are commercially available. The doses used for the injection may be adjusted according to various parameters and according to the aim pursued (labeling, pathology, screening, etc.), the transgene to be expressed, or the duration of expression desired.

In general, the recombinant adenoviruses according to the invention are formulated and administered in the form of doses of between 10⁴ and 10¹⁴ pfu, and, preferably, between 10⁶ and 10¹⁰ pfu. The term pfu (plaque forming unit) corresponds to the infectious power of a viral solution, and is determined by infecting an appropriate cell culture, and measuring the number of plaques of infected cells. The techniques for determining the pfu titer of a viral solution are well known in the art.

The subject of the present invention is, in addition, a method of expressing a transgene of therapeutic interest during which the polynucleotides, cassettes or vectors according to the present invention are used, such that the transgene can be expressed.

Moreover, the invention also relates to any cell modified with a cassette or a vector (e.g., an adenovirus) as described above. The expression “modified” cell is understood to mean any cell containing a polynucleotide or a cassette according to the invention. Modified cells may be intended for implantation into an organism, according to the methodology described in application WO 95/14785. These cells may be, for example, human cardiac cells.

The present invention also relates to transgenic animals, for example, mice carrying a polynucleotide or a cassette as defined above in which the gene encoding the protein of therapeutic interest is replaced with a reporter gene. Such transgenic mice may be used to screen molecules for their activity on the regulatory sequences of the gene encoding the CARP protein. Molecules may be administered to mice and, after sacrificing, histological sections may be prepared in order to identify the tissues stained with the reporter gene.

The transgenic animals according to the present invention also constitute molecular biology study means for understanding the molecular mechanisms underlying cardiac pathologies of genetic origin, such as cardiac insufficiency, cardiac hypertrophy, cardiac hyperplasia, and myocardial infarction. By way of example, there may be mentioned murine models for studying myocarditis in which the gene encoding interferon-1 (IFN-1) is inactivated (Aitken et al., Circulation, 90 (1994) 1-139).

Other animal models of interest according to the present invention may comprise the polynucleotide according to the invention linked to transgenes such as protooncogenes or oncogenes, for example, c-myc, thus constituting models of hyperplasia (Jackson et al., Mol Cell Biol, 10 (1990) 3709-3716), p21-ras for models of ventricular hypertrophy (Hunter et al., J Biol Chem, 270 (1995) 23176-23178), and the nuclear antigen of the Epstein-Barr virus for studying certain cardiomyopathies (Huen et al., J Gen Virol, 74 (1993) 1381-1391).

According to another embodiment, the transgenic animals according to the invention are experimental models of cardiac hypertrophy and comprise an expression cassette in which the transgene encodes for example calmodulin (Gruver et al., Endocrinology, 133 (1993) 376-388), interleukin-6 or the interleukin-6 receptor (Hirota et al., Proc Natl Acad Sci. USA, 92 (1995) 4862-4866), cardiotrophin-1 (Pennica et al., Proc Natl Acad. Sci. USA, 92 (1995) 1142-1146), and, finally, the α-adrenergic receptor (Milano et al., Proc Natl Acad. Sci. USA, 92 (1994) 10109-10113).

Additionally, the polynucleotides according to the invention, modified to allow an increase in the expression of the CARP gene, also form part of the invention. The transgenic animals thus obtained constitute experimental tools for myocardial infarction (Stanton et al., Circul Res, 86 (2000) 939-945).

To carry out the present invention, a person skilled in the art can advantageously refer to the following manual: Sambrook et al. (Molecular Cloning, A Laboratory Manual, Cold Spring Harbor Laboratory Press, New York 1989), or one of its recent editions.

The present invention is described in greater detail with the aid of the following examples, which should be considered as illustrative and nonlimiting.

### LEGENDS TO THE FIGURES

FIG. 1: illustrates the nucleotide sequence (SEQ ID NO: 1) of the polynucleotide upstream of the gene encoding the mouse CARP protein;

FIG. 2: illustrates the nucleotide sequence (SEQ ID NO: 2) of the polynucleotide upstream of the gene encoding the human CARP protein;

FIG. 3: illustrates the nucleotide sequence of the polynucleotide located between positions −2702 and +38 (SEQ ID NO: 3), relative to transcription start position +1 of the human CARP gene;

FIG. 4: illustrates the nucleotide sequence of the polynucleotide located between positions −2108 and +38 (SEQ ID NO: 4), relative to transcription start position +1 of the human CARP gene;

FIG. 5: illustrates the nucleotide sequence of the polynucleotide located between positions −2011 and +38 (SEQ ID NO: 5), relative to transcription start position +1 of the human CARP gene;

FIG. 6: illustrates the nucleotide sequence of the polynucleotide located between positions −1543 and +38 (SEQ ID NO: 6), relative to transcription start position +1 of the human CARP gene;

FIG. 7 illustrates the nucleotide sequence of the polynucleotide located between positions −772 and +38 (SEQ ID NO: 7), relative to transcription start position +1 of the human CARP gene;

FIG. 8: is a schematic representation of the plasmid pXL3634;

FIG. 9: is a schematic representation of the plasmid pXL3728;

FIG. 10: is a schematic representation of the plasmid pXL4254;

FIG. 11: is a schematic representation of the plasmid pXL4253;

FIG. 12: is a schematic representation of the plasmid pXL4269;

FIG. 13: is a schematic representation of the plasmid pXL4237;

FIG. 14: is a schematic representation of the plasmid pXL4255;

FIG. 15: is a schematic representation of the plasmid pXL4330;

FIG. 16: is a schematic representation of the plasmid pXL4331;

FIG. 17: is a schematic representation of the plasmid pXL4089;

FIG. 18: is a schematic representation of the plasmid pXL4301;

FIG. 19: is a schematic representation of the plasmid pXL4302;

FIG. 20: is a schematic representation of the plasmid pXL4086;

FIG. 21: illustrates the relative activity in vitro of the plasmids pXL3635 and pXL3634 with respect to the reference activity of the CMV promoter (pRL-CMV). The activity of each promoter is expressed as the Photinus pyralis luciferase activity normalized with the Renilla reniformis luciferase activity.

FIG. 22A: is a schematic representation of the plasmid pXL3759;

FIG. 22B: is a schematic representation of the adenovirus AV1.0 CARP-Luc+;

FIG. 23A: illustrates the luciferase activity (pg luciferase/heart) 7 days after intracardiac transdiaphragmatic injection in rats of variable quantities of plasmids pXL3031 and pXL3634;

FIG. 23B: illustrates the luciferase expression (pg luciferase/heart) 7 days after intracardiac transdiaphragmatic injection in rats hearts of 25 g of plasmids pXL3031 and pXL3635, pXL3130, and pXL3153.

FIG. 24: represents the ratio of the expression of luciferase in the heart relative to the expression in the muscle as a function of the expression in the heart obtained following intracardiac administrations of plasmids pXL3031, pXL3634, pXL3635, pXL3153, and pXL3130.

FIG. 25: illustrates the construction of the various plasmids;

FIG. 26: illustrates the expression of luciferase in differentiated cardiomyocytes following administration of plasmids carrying various 5′ deletions of the human CARP promoter. The liver-specific promoter ApoAII (−911/+59) served as a negative control;

FIG. 27: illustrates the expression of luciferase in differentiated cardiomyocytes following administration of plasmids pXL4089, pXL4301, pXL4302, pXL4330, and pXL4680. The controls were the liver-specific promoter ApoAII (−911/+59), i.e., pXL4086, and the plasmid pXL3031 containing the CMV promoter.

FIG. 28: illustrates the luciferase expression in differentiated cardiomyocytes and MDCK epithelial cells following administration of plasmids pXL4089, pXL4237, pXL4301, and pXL4330. Plasmids pXL4055 and pXL4086 were used as controls.

FIG. 29: illustrates the luciferase expression in SD rats 3 days after intracardiac injection of plasmids pXL4089, pXL4254, pXL4253, pXL4269, pXL4237, and pXL4255. Plasmids pXL4081, pXL4055, and pXL4089 were used as controls.

FIG. 30: illustrates the nucleotide sequence of the human cardiac α-actin 5′UTR (+1/+739) (SEQ ID NO: 8), the nucleotide sequence of the human cardiac α-actin 5′UTR (+1/+739, Δ+119/+645) (SEQ ID NO: 9), which is designated Int1, and the nucleotide sequence of the human GH polyA 3′UTR (SEQ ID NO: 10).

FIG. 31: illustrates the nucleotide sequence of the polynucleotide located between positions −3020 and +38 (SEQ ID NO: 13), relative to transcription start position +1 of the human CARP gene.

### EXAMPLES

### Example 1

### Characterization of the Polynucleotide Upstream of the CARP Gene

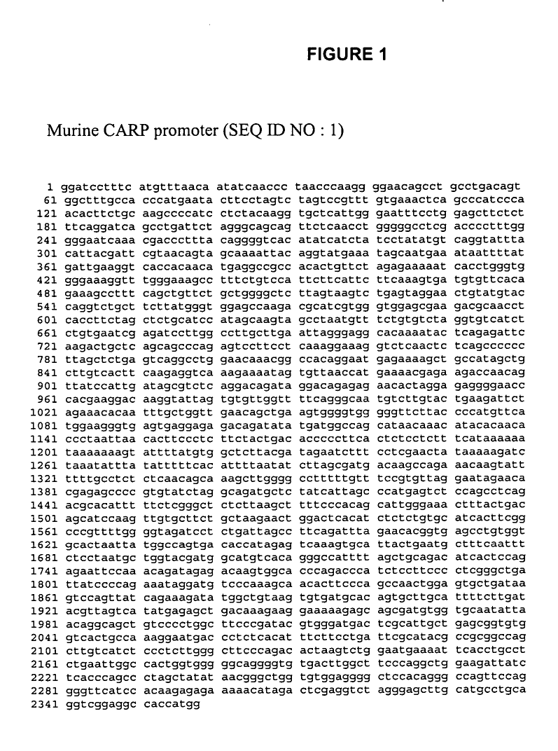

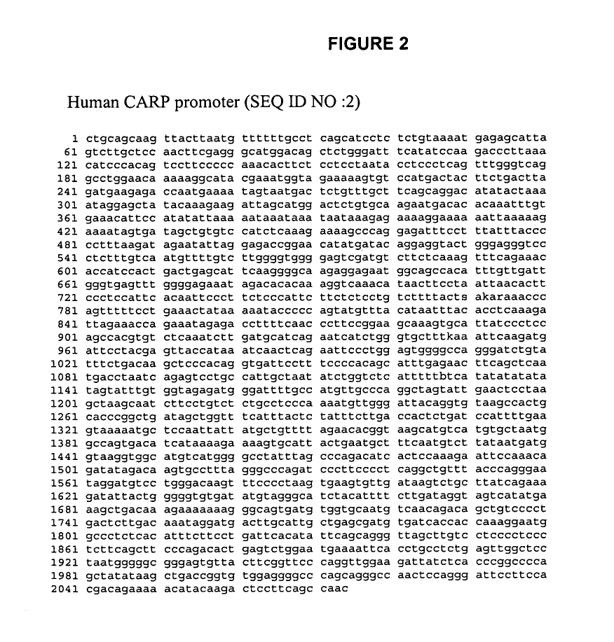

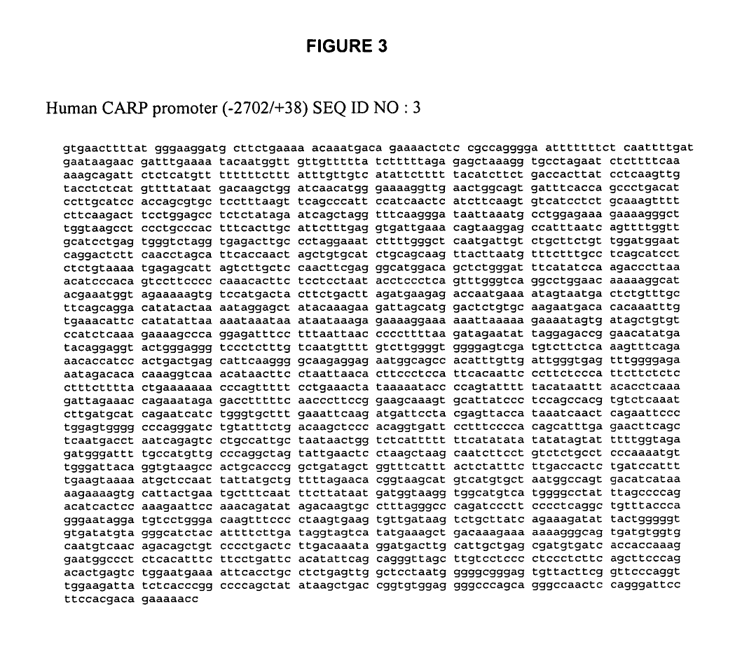

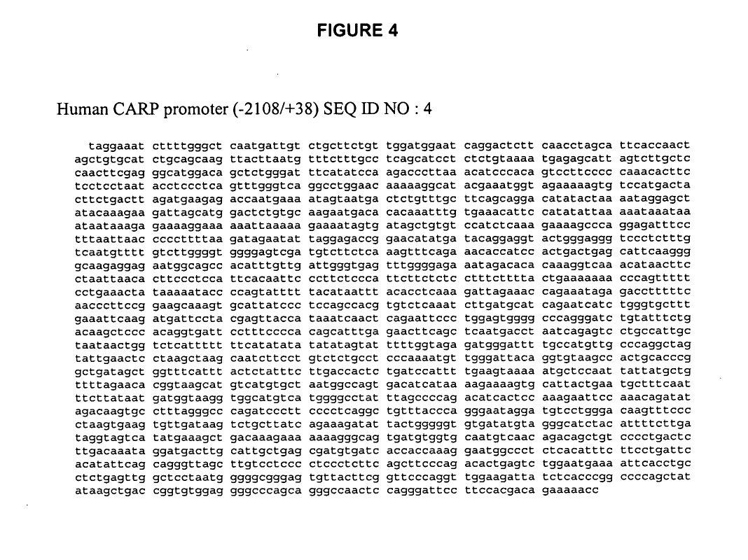

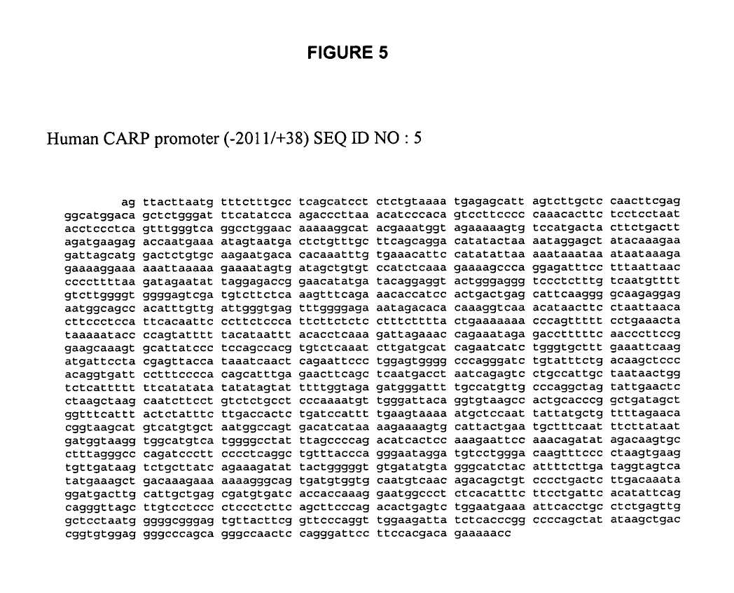

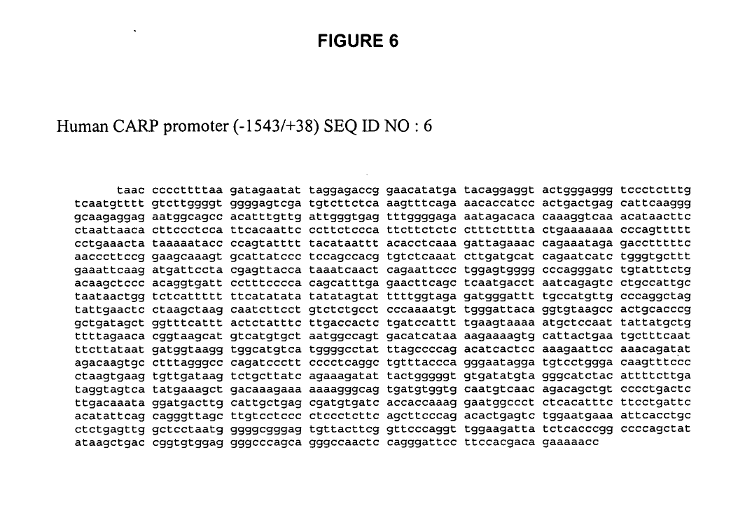

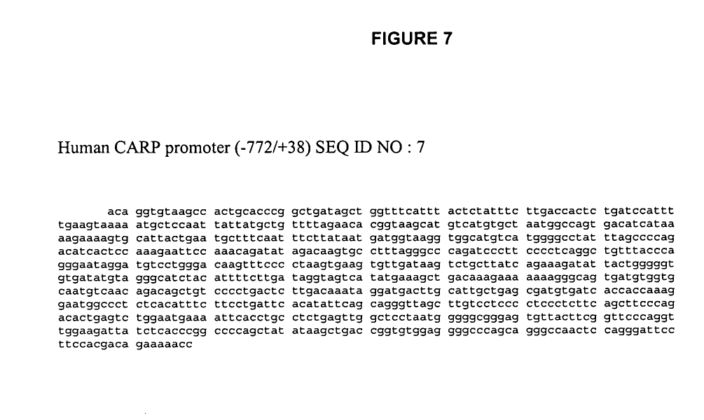

A 2.3 kilobase (kb) BamHI-XhoI fragment of the sequence at the 5′-end of the mouse gene encoding the CARP protein was cloned and sequenced on both strands according to the chain termination method (Sanger et al., 1977, Proc. Natl. Acad. Sci. USA, 74, 5463) using a Sequenase® kit (United States Biochemical, Cleveland, Ohio). The sequence (SEQ ID NO: 1) is represented in FIG. 1 and comprises a portion upstream of the gene encoding the mouse CARP protein between nucleotides −2266 and +92 relative to transcription start position +1.

### Example 2

### Construction of CARP Plasmid Vectors

2.1 Plasmid pXL3634

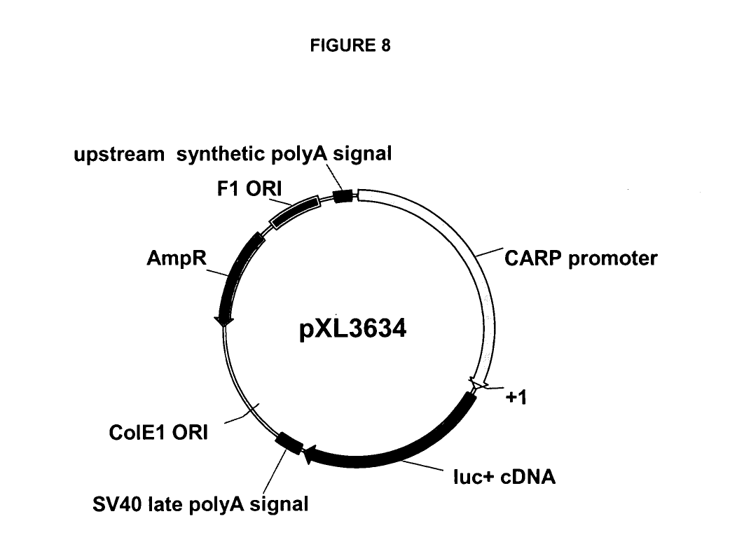

After filling in the BamHI site, the 2.3 kb BamHI-XhoI fragment characterized in Example 1 was cloned into the plasmid pGL3-Basic (Promega), which had been digested with XhoI and SmaI, in order to obtain the plasmid pXL3634. A schematic representation of this plasmid is presented in FIG. 8.

2.2 Plasmid PXL3728

The plasmid pXL3728 was obtained from the plasmid pXL3179, which was derived from the plasmid pXL2774 (WO 97/10343) in which the gene encoding a fusion between the signal peptide of human fibroblast interferon and the cDNA of FGF1 (fibroblast growth factor 1) (sp-FGF1; Jouanneau et al., Proc. Natl. Acad. Sci USA 88 (1991), 2893-2897) was introduced under the control of the promoter obtained from the human cytomegalovirus early region (hCMV IE) and the polyadenylation signal of the SV40 virus late region (GenBank SV4CG).

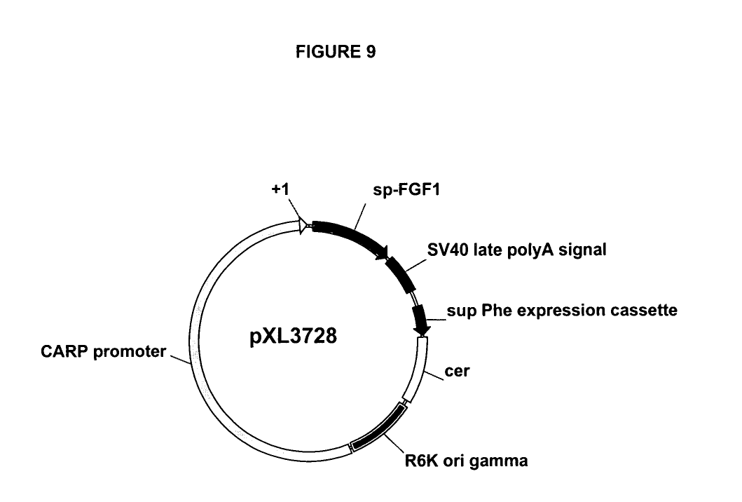

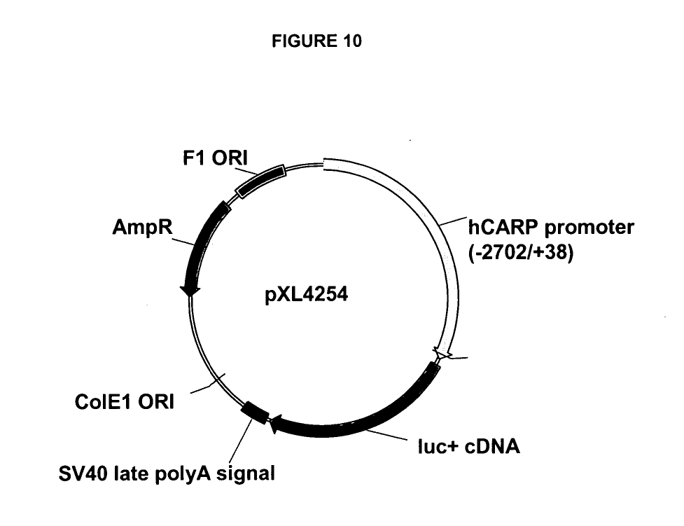

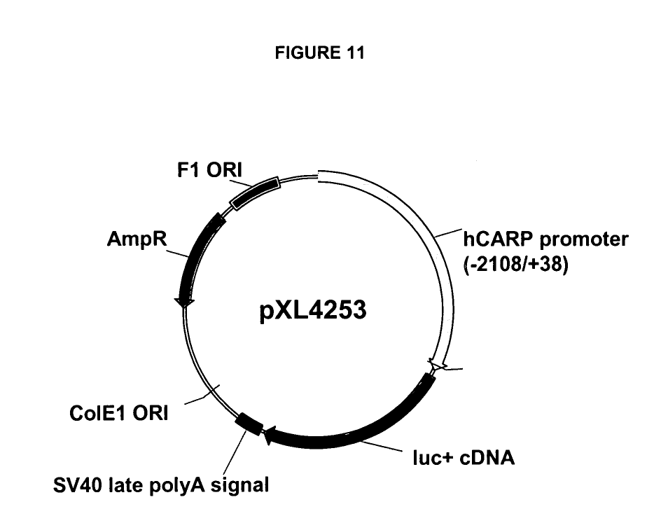

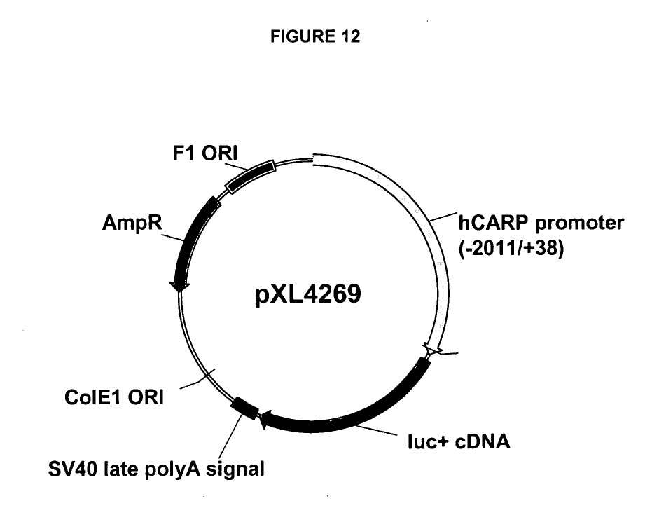

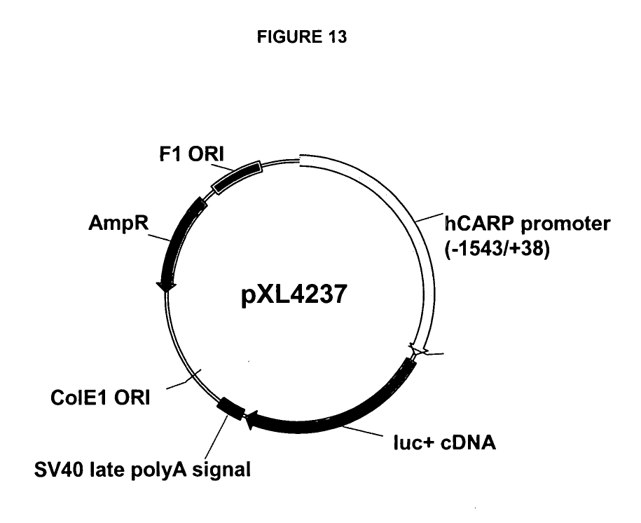

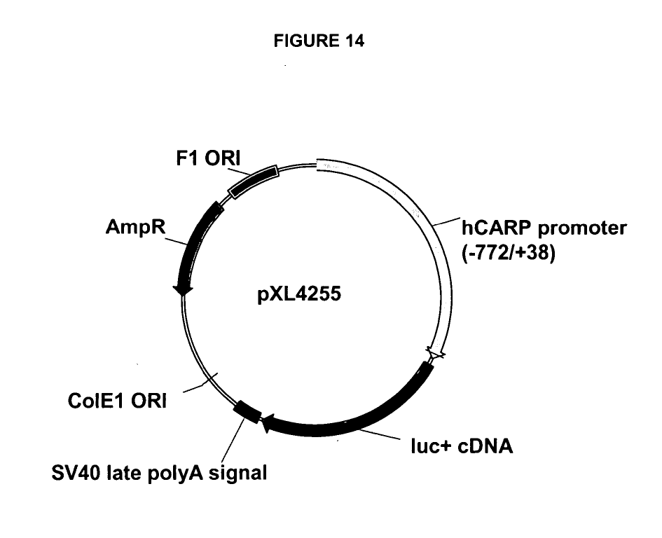

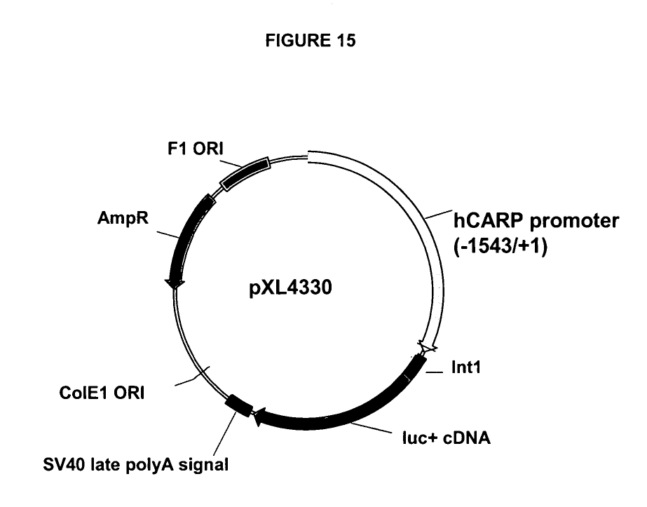

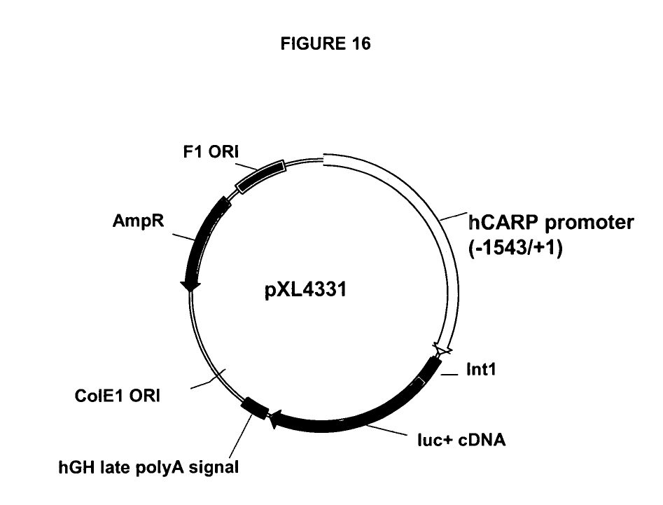

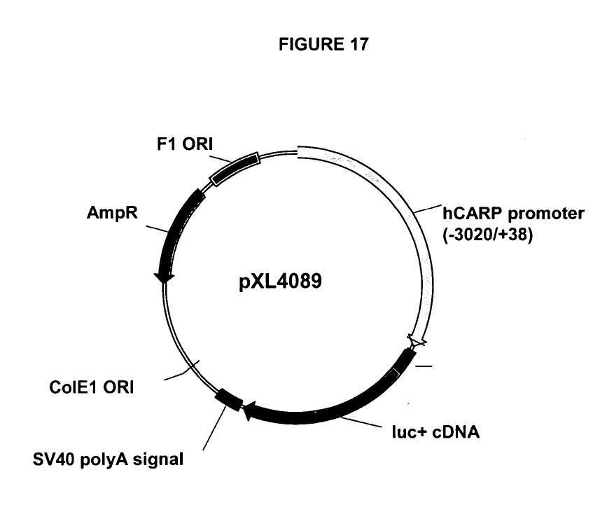

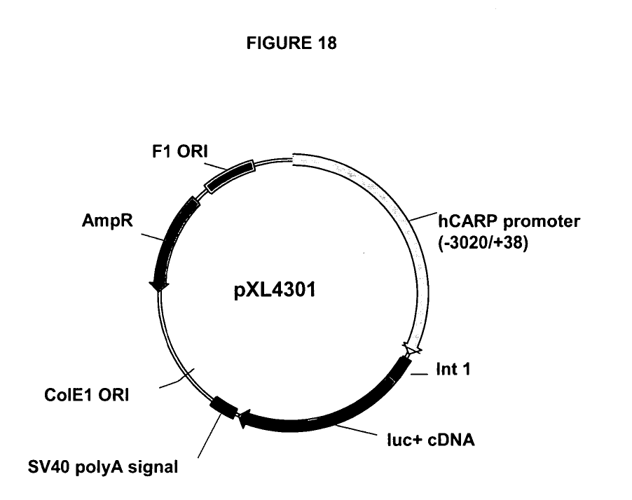

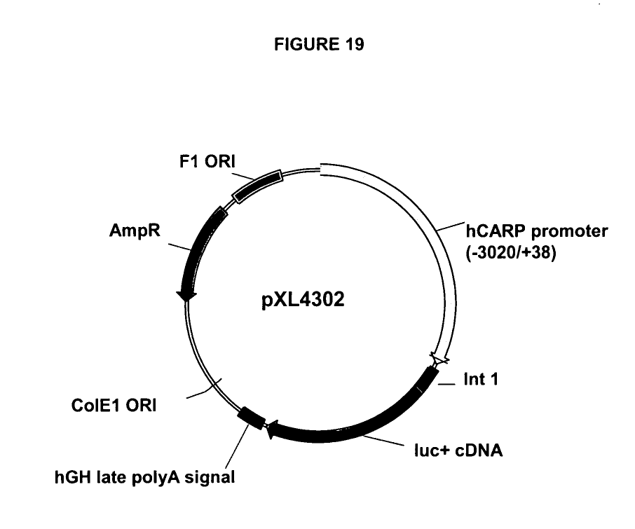

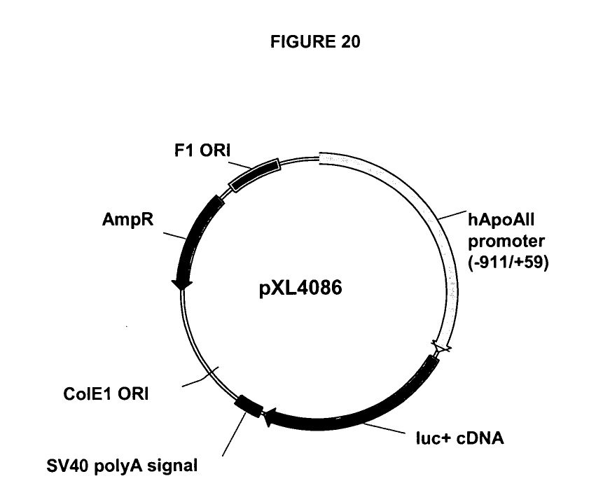

The 2.3 kb BamHI-XhoI fragment characterized in Example 1, whose ends have been filled in, was cloned into the plasmid pXL3179 ((plasmid with conditional origin of replication) PCOR CMV-FGF), previously digested with XbaI and EcORI, in order to obtain the plasmid pXL3728. A schematic representation of this plasmid is presented in FIG. 9.

2.3 Plasmid pXL3729

An EcORI-SalI fragment of the plasmid pXL3634 was cloned into the plasmid pXL3728, which had been previously digested with EcORI-SalI in order to obtain the plasmid pXL3729.

2.4 Plasmid pXL4089

The fragment −3020/+38 of the human CARP gene (SEQ ID NO: 13) was cloned into the plasmid pXL3728, which had been previously digested in order to obtain the plasmid pXL4089.

2.5 Plasmid DXL4301

The fragment −3020/+38 of the human CARP gene (SEQ ID NO: 13) and the Int1 sequence corresponding to the 5′-UTR of the cardiac α-actin gene (+1/+739, Δ+119/+645) (SEQ ID NO: 9) were cloned into the previously digested plasmid pXL3728 in order to obtain the plasmid pXL4301.

2.6 Plasmid pXL4302

The fragment −3020/+38 of the human CARP gene (SEQ ID NO: 13), the Int1 sequence corresponding to the 5′-UTR of the cardiac α-actin gene (+1/+739, Δ+119/+645) (SEQ ID NO: 9), and the 3′UTR of hGH polyA (SEQ ID NO: 10), were cloned into the previously digested plasmid pXL3031 in order to obtain the plasmid pXL4302.

2.7 Plasmid pXL4254

The fragment −2702/+38 of the human CARP gene (SEQ ID NO: 3), and the 3′-UTR of SV40 late polyA, were cloned into the previously digested plasmid pXL3031 in order to obtain the plasmid pXL4254.

2.8 Plasmid pXL4253

The fragment −2108/+38 of the human CARP gene (SEQ ID NO: 4), and the 3′-UTR of SV40 late polyA, were cloned into the previously digested plasmid pXL3031 in order to obtain the plasmid pXL4253.

2.9 Plasmid pXL4269

The fragment −2011/+38 of the human CARP gene (SEQ ID NO: 5), and the 3′-UTR of SV40 late polyA, were cloned into the previously digested plasmid pXL3031 in order to obtain the plasmid pXL4269.

2.10 Plasmid pXL4237

The fragment −1543/+38 of the human CARP gene (SEQ ID NO: 6), and the 3′-UTR of SV40 late polyA, were cloned into the previously digested plasmid pXL3031 in order to obtain the plasmid pXL4237.

2.11 Plasmid pXL4255

The fragment −772/+38 of the human CARP gene (SEQ ID NO: 7), and the 3′-UTR of SV40 late polyA, were cloned into the previously digested plasmid pXL3031 in order to obtain the plasmid pXL4255.

2.12 Plasmid pXL4330

The fragment −1543/+1 of the human CARP gene (SEQ ID NO: 6), the Int1 sequence corresponding to the 5′-UTR of the cardiac α-actin gene (+1/+739, Δ+119/+645) (SEQ ID NO: 9), and the 3′-UTR of SV40 late polyA, were cloned into the previously digested plasmid pXL3031 in order to obtain the plasmid pXL4330.

2.13 Plasmid pXL4331

The fragment −1543/+1 of the human CARP gene (SEQ ID NO: 6), the Int1 sequence corresponding to the 5′-UTR of the cardiac α-actin gene (+1/+739, Δ+119/+645) (SEQ ID NO: 9), and the 3′UTR of hGH late polyA (SEQ ID NO: 10), were cloned into the previously digested plasmid pXL3031 in order to obtain the plasmid pXL4331.

### Example 3

### Comparative Plasmids

3.1 Plasmids pXL3130 and pXL3153

Plasmids pXL3130 and pXL3153 contain, respectively, the human smooth muscle α-actin promoter (−680 to +30) and the mouse SM22 promoter (−436 to +43) coupled to the CMV enhancer (−522 to −63) as described in application WO 00/18908.

3.2 Plasmid pXL3635

The RSV −229 to +34 promoter was cloned from a construct containing a longer version of the RSV promoter (contained in Ad1.0RSVLAcZ, Stratford-Perricaudet et al., J Clin Invest 90 (1992) 626-30) by PCR using of the primers 5′-GGC GAT TTA MT MT GTA GTC TTA TGC MT-3′ (SEQ ID NO: 11) and 5′-GGG GTC TAG MG GTG CAC ACC MT GTG GTG A-3′ (SEQ ID NO: 12), which introduce, respectively, an SwaI and XbaI site at the 5′- and 3′-ends of the PCR fragment. These two restriction sites were then used to introduce the promoter fragment into pGL3-basic to generate pXL3635.

3.2 Plasmid pXL3031

The plasmid pXL3031 is described by Soubrier et al., Gene Ther. 6 (1999), 1482-8. It is a vector derived from the plasmid pXL2774 (WO 97/10343) in which the luc gene encoding the modified Photinus pyralis luciferase (cytoplasmic) obtained from pGL3basic (GenBank: CVU47295) was introduced under the control of the promoter obtained from the human cytomegalovirus early region (hCMV IE, GenBank HS51EE) and of the polyadenylation signal of the SV40 virus late region (GenBank SV4CG).

3.3 Plasmid pXL4086

The fragment −911/+59 of the human ApoAII gene and the 3-′UTR of SV40 late polyA, were cloned into the previously digested plasmid pXL3031 in order to obtain the plasmid pXL4086.

### Example 4

### Cell Cultures

In order to establish primary cultures of rat cardiomyocytes, pregnant rats were killed in a chamber saturated with CO₂. After opening the abdomen, the uterine horns were removed and washed in PBS at room temperature. The embryos were released from their envelopes and the placenta cut (10 to 12 embryos per rat). The hearts were removed and washed in phosphate-buffered saline (PBS)/glucose. Under a binocular lens, the auricles and large vessels were removed, and then the hearts were again cleaned in ADS/glucose so as to retain only the ventricles, which were then rinsed 3 times in sterile ADS/glucose.

The hearts were then trypsinized in 0.3 ml of an ADS/glucose/trypsin mixture per heart, using trypsin T 4674 (Sigma, St. Louis, Mo.) at a final concentration of 0.1 mg/ml, for 20 min at 37° C., with gentle stirring (60 to 100 revolutions per min).

The supernatant was removed and the trypsin was inactivated by adding 1 ml of decomplemented fetal calf serum (FCS). After centrifugation at 1500 rpm for 10 minutes, the supernatant was removed and the cardiac cells were taken up in 1 ml of decomplemented FCS. In parallel, the steps of treating with trypsin were repeated 5 to 6 times until complete dissociation of the cells was obtained. The pool of cells was centrifuged at 1500 rpm for 10 minutes, then washed twice in FCS and the cells were finally collected on a grid filter.

The cells thus separated were placed in culture at a concentration of 10⁶ cells/well for a 24-well plate or at a concentration of 2×10⁶ cells/well for a 12-well plate. Each well contained 1 ml of culture medium.

The culture medium comprises, for a total volume of 100 ml, 68 ml of Dulbecco's Modified Eagle's Medium (DMEM) (without pyruvate) (Gibco-BRL), 17 ml of M199 (Sigma M 4530), 10 ml of decomplemented horse serum (Sigma H6762), 5 ml of decomplemented FCS (Gibo-BRL) and 1 ml of 100×Pen/Strep/glutamine mixture (Gibco-BRL).

The cardiomyocytes were cultured for a period of about 1 or 2 days.

### Example 5

### Cell Transfection

### Example 5.1

### Primary Cardiomyocyte Cultures

Primary cultures of cardiomyocytes were cotransfected with 1 ng of pRL-CMV (Promega Inc., Madison, Wis.), various amounts of pXL3635 and pXL3634 (1-100 ng each), and various amounts of pUC19 g for a total of 500 ng of DNA per well.

The mixture of plasmids was incubated with 6 nmol of RPR 120535B (Byk et al., J Med Chem. 41 (1998) 229-35) per μg of DNA (i.e., 0.3 μl of 10 mM lipid) in a final volume of 20 μl of 150 mM NaCl and 50 mM bicarbonate. The mixture was then vortexed for 5 seconds and incubated for an additional 20 to 30 minutes at room temperature.

The plasmid/lipid mixture was added to 250 μl of serum-free medium and incubated with the cells for at least 2 hours. The medium was removed and the cells were incubated for 24 hours to 7 days at a temperature of 37° C. in the presence of 5% CO₂. The cells were harvested at either 24 or 48 hours after transfection, and the Renillia luciferase and Firefly luciferase activities were analyzed with the Promega Dual Luciferase® kit according the manufacturer's instructions.

### Example 5.2

### Differentiated Cardiomyocytes (H9C2)

A cardio-specific subclone (H9C2#25) of H9C2 cardiomyoblastic cells was plated at 10⁴ cells per well in 24-well plates the day before transfection. Cells were transfected using Lipofectamine 2000™ reagent (InVitrogen) at a ratio of 1 μg of DNA per 1 μl of Lipofectamine™. Cells were transfected in triplicate with 3, 10, or 30 ng of each plasmid. The total amount of transfected DNA was 500 ng per well and transfection was normalized by adding pSL301 plasmid. Along with the other plasmids, the cells were transfected with 1 ng of pCMV-RenillaLuc as an internal control for transfection efficiency. The cells were differentiated to cardiomyocytes following transfection by replacing the proliferating medium with differentiating medium (low serum). Luciferase activity was measured in differentiated cardiomyocytes.

### Example 5.3

### Kidney Epithelial Cells (MDCK)

MDCK cells were plated at 6×10⁴ cells per well (24well-plates) and transfected the following day using Lipofectamine 2000™ reagent (InVitrogen) according to the manufacturer's instructions. The total amount of transfected DNA was 1000 ng and transfection was normalized by adding pSL301 plasmid. Along with the other plasmids, the cells were transfected with 1 ng of pCMV-RenillaLuc as an internal control for transfection efficiency.

### Example 6

### Comparative Evaluation of the In Vitro Activity of the Polynucleotide

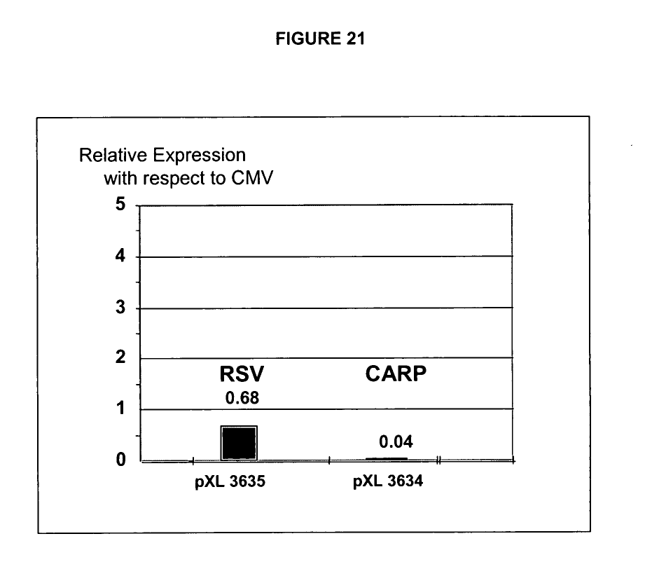

The relative promoter activities of a CARP polynucleotide (pXL3634) and an RSV (pXL3635) promoter were evaluated in vitro by transient transfection of primary cultures of rat cardiomyocytes, and were expressed relative to the activity of pRL-CMV (FIG. 21). The results show that the polynucleotide upstream of the CARP gene (pXL3634) has very low in vitro activity (i.e., approximately 0.04% of the CMV promoter activity). The relative activity of the nonspecific, strong RSV promoter (pXL3635) was also low (i.e., approximately 0.68% of the CMV promoter activity).

### Example 7

### Comparative Evaluation of the Activity of the Polynucleotide in Differentiated Cardiomyocytes H9C2

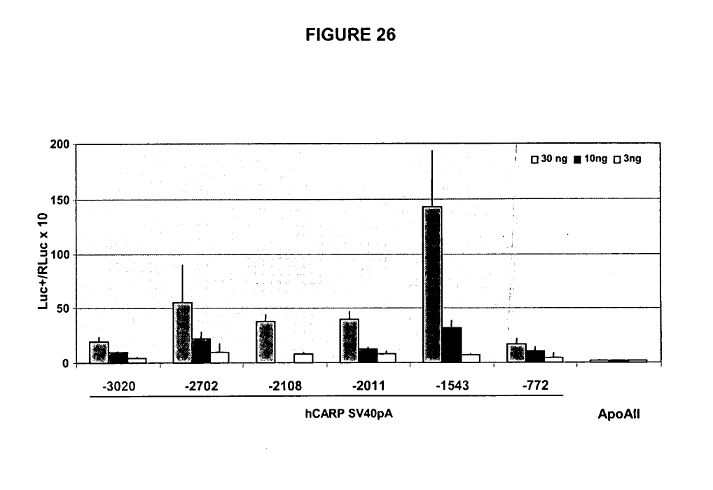

The various plasmids described in the Examples above comprise either a complete or 5′-deleted CARP promoter, murine and human cardiac α-actin promoters, or a liver-specific human ApoAll promoter and a CMV promoter as controls (FIG. 26). All plasmids contain an expression cassette for the modified firefly (Photinus pyralis) luciferase reporter gene (luc+). This expression cassette was inserted into an original backbone PCOR, as described in U.S. Patent Publication Nos: US2001/014476 and US2003/161844, thereby creating a closed circular plasmid. The late polyadenylation signal from Simian Virus 40 or from human GH is inserted downstream of the luciferase gene.

Luciferase activity was measured according to the manufacturer's instructions (Dual-Luciferase® reporter assay (Promega)) and quantified by luminescence counting (relative light units or RLU). For each experiment, data were expressed as the mean of triplicate measurements of luciferase activity. Each experiment was performed twice. The results were analyzed by Multiway ANOVA test and then by the Newman-Keuls method to group plasmids with equivalent activities.

A deletion series of hCARP constructs as described in the above examples was assayed for transcriptional activity in cardiomyocyte cell cultures. The plasmids tested were: pXL4089 (−3020/+38 of hCARP gene, SEQ ID NO: 13), pXL4254 (−2702/+38 of hCARP gene, SEQ ID NO: 3), pXL4253 (−2108/+38 of hCARP gene, SEQ ID NO: 4), pXL4269 (−2011/+38 of hCARP gene, SEQ ID NO: 5), pXL4237 (−1543/+38 of hCARP gene, SEQ ID NO: 6), and pXL4255 (−772/+38 of hCARP gene, SEQ ID NO: 7). The plasmid pXL4086, which contains the ApoAII (−911/+59) liver-specific promoter, was used as a negative control.

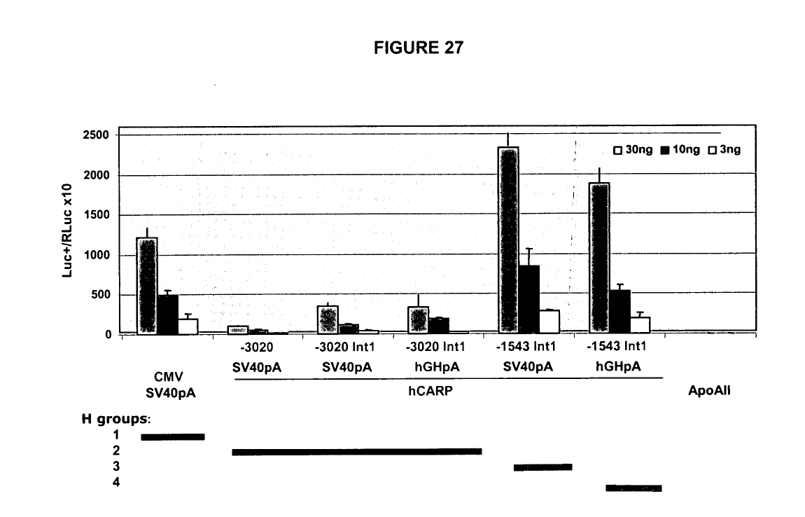

FIG. 27 shows a representative analysis performed in triplicate for each reporter construct. Error bars indicate standard deviation. The results showed that the hCARP −1543Luc+ construct (pXL4237) had the highest transcriptional activity. Its activity was significantly higher activity than the vector hCARP −3020/Luc+ (pXL4089). However, deletion of the region −1543/−772 on the hCARP 5′ flanking region to give the hCARP −772Luc+ (pXL4255) construct resulted in a considerable reduction in activity. Therefore, the hCARP −1543Luc+ construct increases expression and decreases the total length of the vector, preserving the strength of the promoter.

The above 5′ deleted human CARP promoter constructs were tested for their transcriptional activity in cardiomyocyte cell cultures in combination with different post-transcriptional regulatory elements. In particular, the various 5′ deleted constructs were tested in combination with the 5′UTR Int1 (SEQ ID NO: 9) and either the 3′UTR of the human GH gene (SEQ ID NO: 10) or the 3′UTR of SV40. The plasmids tested were: pXL4089 (−3020/+38 of hCARP gene with SV40 polyA), pXL4301 (−3020/+38 of hCARP gene with Int1 and SV40 polyA), pXL4302 (−3020/+38 of hCARP gene (SEQ ID NO: 13) with Int1 (SEQ ID NO: 9) and hGH polyA (SEQ ID NO: 10)), pXL4330 (−1543/+38 of hCARP gene (SEQ ID NO: 6) with Int1 (SEQ ID NO: 9) and SV40 poly A), and pXL4331 (−1543/+38 of hCARP gene (SEQ ID NO: 6) with Int1 (SEQ ID NO: 9) and hGH polyA (SEQ ID NO: 10)). The luciferase activities driven by the CMV(−522/+72) promoter (pXL3031) and a liver-specific promoter ApoAII (−911/+59) were also measured as positive and negative controls, respectively. The data shown in FIG. 27 are a representative analysis performed in triplicate for each transfected reporter construct. Error bars indicate standard deviation.

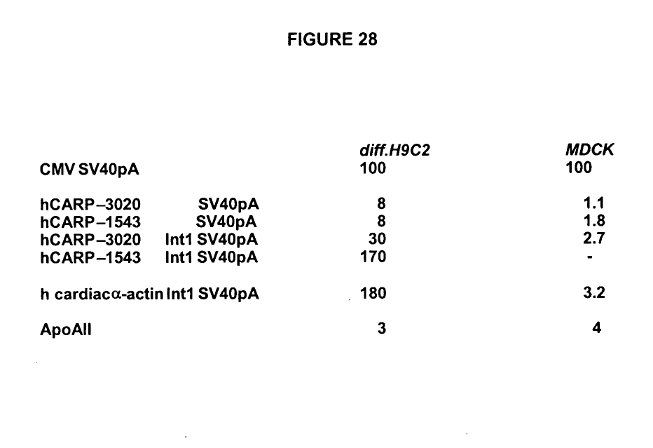

FIG. 28 shows that both hCARP Int1 Luc+ constructs had higher transcriptional activity than other hCARP constructs or the CMV vector (pXL3031). The data show a synergistic effect of the 5′UTR Int1 and the truncated −1543 hCARP promoter on luciferase expression. The 3′ UTR of the human GH gene cloned in the hCARP constructs did not increase the activity of these vectors as compared to results obtained with constructs containing the 3′UTR of SV40.

Both hCARP Int1 Luc+vectors provide a substantial elevation in the level of luciferase expression in differentiated cardiomyocytes by virtue of inclusion of a new 5′UTR named Int1 and partial deletion of the promoter.

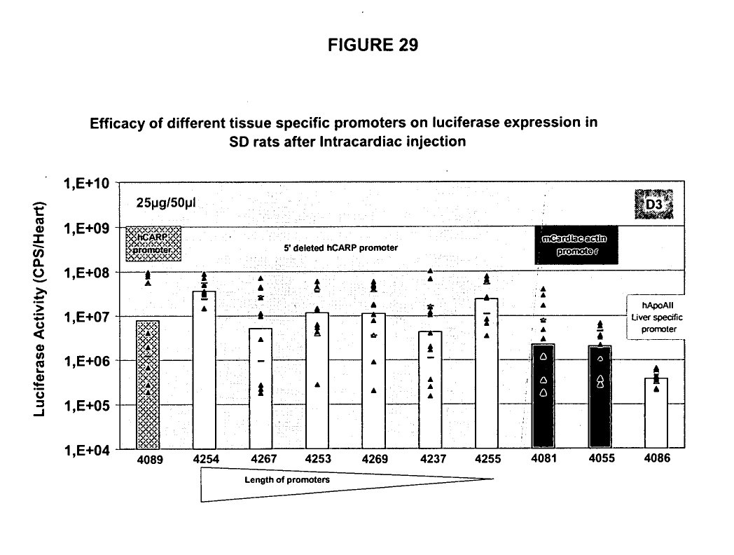

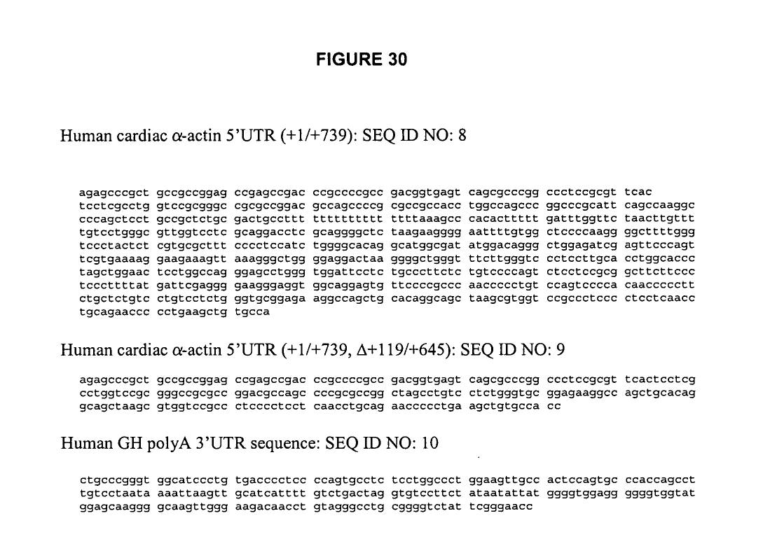

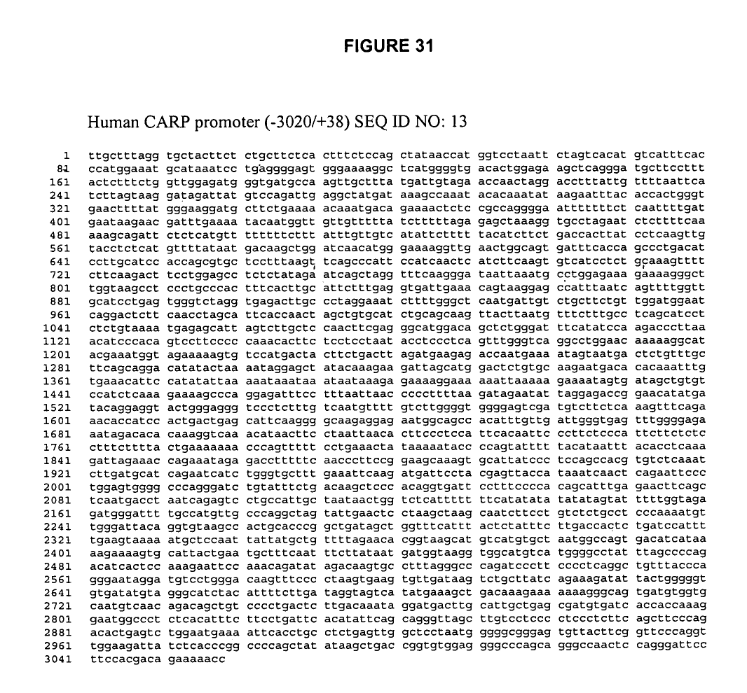

The tissue specificity of the promoters derived from the human CARP and cardiac α-actin genes was also assessed. The transcriptional activity of the hCARP and human cardiac α-actin constructs was measured in transiently transfected cardiomyocytes and in MDCK epithelial cells. Positive control (CMV) and negative control (liver-specific promoter ApoAII) activity was also measured (FIG. 28). The data for each construct were derived from at least 4 independent experiments performed in triplicate. To facilitate direct comparison between cell types, data were expressed as the measured RLU from each construct relative to that obtained from the CMV construct, which was assigned a value of 100. FIG. 29 shows that the hCARP and human cardiac α-actin plasmids exhibit in vitro cardiac muscle-specific activities.

### Example 8

### Construction of an Adenovirus

An adenovirus that allows the expression of luciferase under the control of the CARP promoter was constructed according to the method of Crouzet et al. (Proc. Natl. Acad. Sci. USA, 94 (1997) 1414-1419). The expression cassette was identical to that of plasmid pXL3634 (FIG. 8).

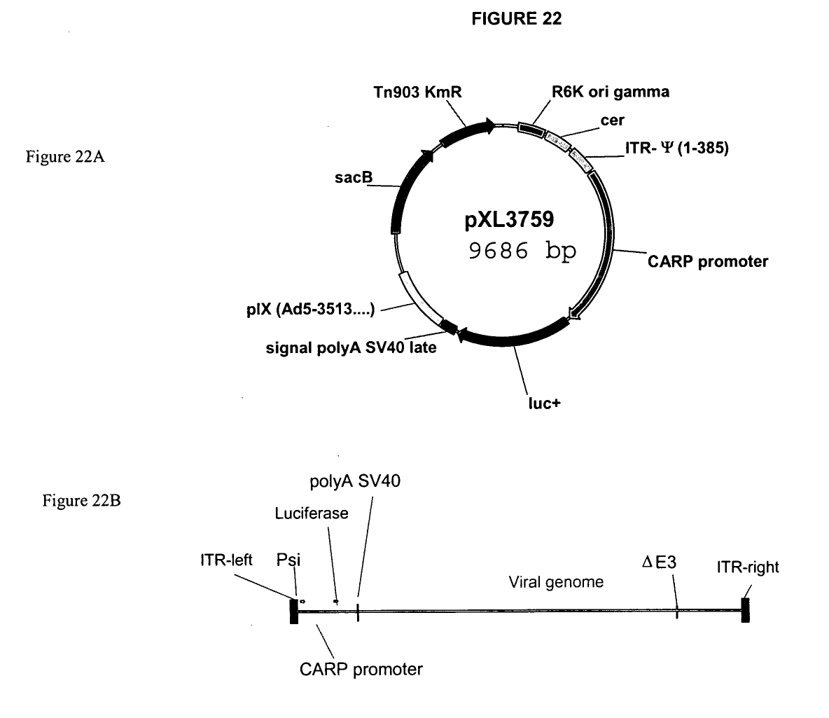

A shuttle vector, which allows recombination in Escherichia coli, was constructed in two stages. First, the hCARP promoter (fragment: XhoI filled with Klenow/BamHI) was introduced into pXL3474 (digested with ScaI and BglII) between the regions ITR- and pIX in order to generate the plasmid pXL3758. Plasmid pXL3759 (FIG. 22A) was then generated by introducing the fragment containing the luciferase cDNA and the SV40 polyadenylation site (BamHI fragment filled with Klenow/BstEII of pXL3634) into pXL3758 (which had been digested with BstBII (filled in with Klenow) and BstEII).

Homologous double recombination in E. coli was accomplished as described above, against a plasmid pXL3215 containing an E1/E3 adenoviral genome into which an RSV-LacZ expression cassette had been introduced into the E1 region. The plasmid pXL3215 is a derivative of the plasmid pXL2689, which contains the origin of replication of the plasmid RK2 and the tetracycline resistance gene (Crouzet et al. Proc. Natl. Acad. Sci. USA, 94 (1997) 1414-1419). The product of this double recombination, the plasmid pXL3778, was verified by sequencing the expression cassette. After release of a linear viral genome by cleavage with PacI, the plasmid was transfected into the Per.C6 cell line (WO 97/00326) to generate the virus AV1.0CARP-Luc+.

The virus was also verified by sequencing the expression cassette and by restriction analysis. The presence of RCA E1+ (replication competent adenovirus) particles was confirmed by nucleic acid hybridization.

Stocks containing a high viral titer were obtained by amplification of the virus in the Per.C6 line, and viral particles were purified on a CsCl gradient. The titer of the virus (measured as viral particles/ml (vp/ml)) was obtained by chromatography. Viral activity was assayed in vitro by titration of luciferase activity after infection of skeletal or cardiac muscle cells and comparison with a control virus comprising a CMV promoter.

### Example 9

### Injection of DNA In Vivo

CD SPRAGUE (SD) rats (200 g) were anesthetized by intraperitoneal injection (1 ml/kg) of a mixture of ketamine (70 mg/ml) and xylazine (6 mg/ml).

Intramyocardiac injections were administered transdiaphragmaticly with a 100 μl Hamilton glass syringe (connected to a Steriflex catheter (ref. 167.10 G19 V) with a stop flange and a BD 26G*3.8, short-bezel needle) after laparotomy. Fifty microliters of DNA solution in 0.9% NaCl were injected over 5 seconds.

After sacrificing the animals, the hearts were removed, rinsed in 0.9% NaCl solution and macroscopically examined. The hearts were homogenized (Ultra-thurax, Diax600 Heidolph) in lysis buffer (luciferase activity kit from Promega E151A) supplemented with protease inhibitors (Cmplete, Roche Diagnostics), and centrifuged for 20 minutes at 4000 rpm at 4° C. Luciferase activity was measured in a LUMAT LB 9501 luminometer after adding 50 μl of Promega luciferase substrate to 10 μl of heart supernatant). Luciferase activities were expressed as pg luciferase/heart using the method described in Mir et al (Proc. Natl. Acad. Sci. USA 96 (1999), 4262-4267).

Alternatively, the hearts were fixed in 3.7% paraformaldehyde and analyzed by immunohistochemistry for the expression of FGF-1.

### Example 10

### Comparative Evaluation of the In Vivo Activity of the CARP Polynucleotide

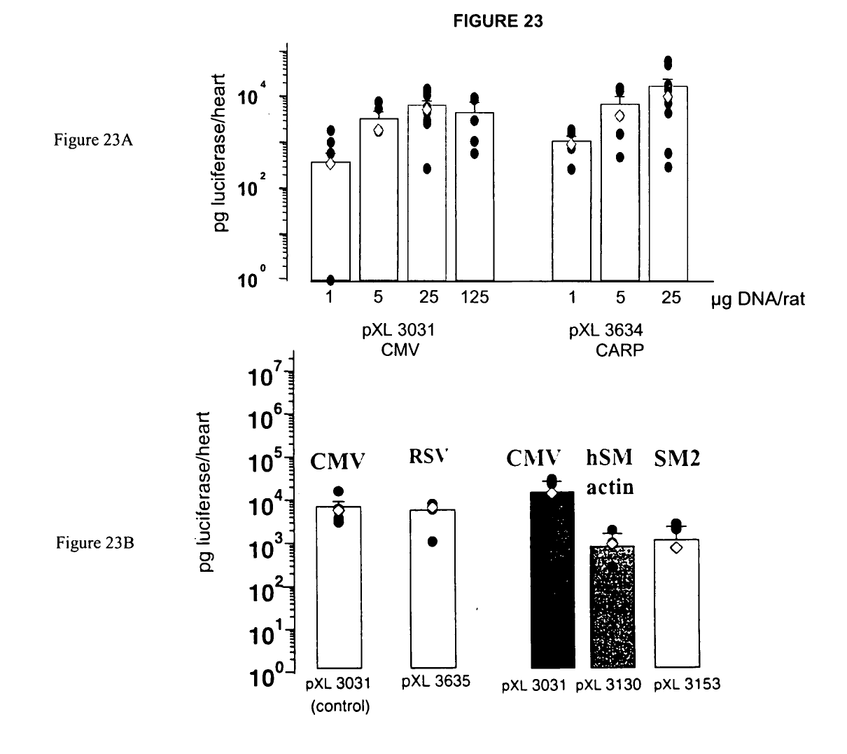

The results presented in FIG. 23A show that the luciferase activities obtained following injection of increasing amounts (i.e., 1, 5, 25 and 125 μg) of plasmids pXL3031 and pXL3634 were not significantly different. Therefore, the polynucleotide upstream of the CARP gene is capable of inducing high levels of in vivo gene expression that are equivalent to those induced by the strong promoter CMV.

In contrast, the expression obtained with another strong viral promoter (i.e., the RSV promoter (pXL3635)) was weaker than that obtained with either the CMV promoter or the polynucleotide upstream of the CARP gene (FIG. 23B).

Moreover, the addition of the CMV enhancer upstream of smooth muscle cell promoters (SM α-actin, pXL3130 or SM22, pXL3153), although demonstrated to be highly efficient in vitro (WO 00/18908), appeared to be ineffective in cardiac cells in vivo (FIG. 23B).

### Example 11

### Evaluation of the Specificity of Expression of the CARP Polynucleotide

Twenty-five micrograms of plasmids pXL3634, pXL3435, or pXL3031 were administered to rats by intracardiac transdiaphragmatic injection.

Ten micrograms of each plasmid was also administered to groups of mice by intramuscular injection into the cranial tibial muscle with or without electrotransfer.

The expression of luciferase was analyzed as described (Mir et al., Proc. Natl. Acad. Sci. USA 96 (1999), 4262-4267) seven days after the injection.

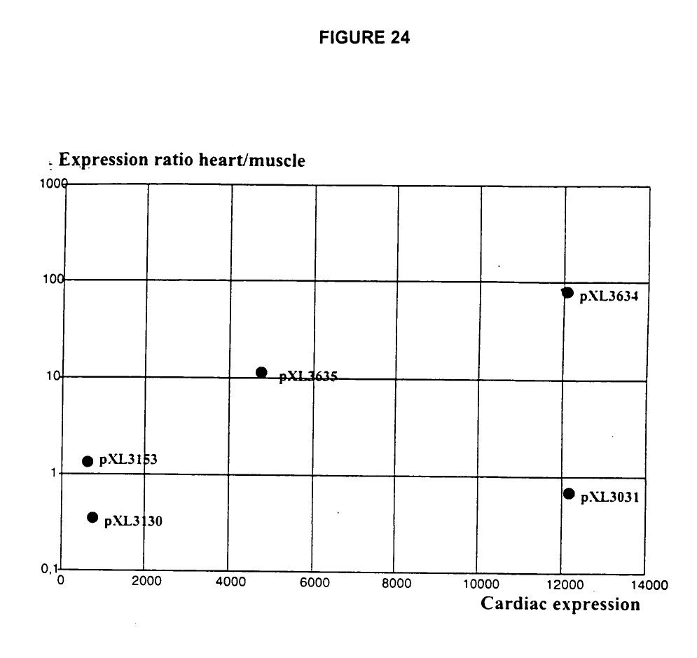

The levels of luciferase expression in the heart were expressed relative to the levels observed in the cranial tibial muscle (FIG. 24).

The results showed that the polynucleotide upstream of the CARP gene and the CMV promoter were the only two promoters capable of inducing the highest expression in the cardiac tissue. However, the heart/muscle expression ratio was 1 with the CMV promoter, whereas this ratio was close to 100 when the polynucleotide upstream of the CARP gene was used, which shows the very high selectivity of the latter for cardiac tissue.

The cardiac-specific expression driven by the polynucleotide of the invention was superior to that of other constructs comprising a smooth muscle cell-specific enhancer and promoter (for example, genes encoding SM-22 and actin) (FIG. 24).

### Example 12

### Evaluation of the Activity and Specificity of the 5′ Deleted Human CARP Promoter

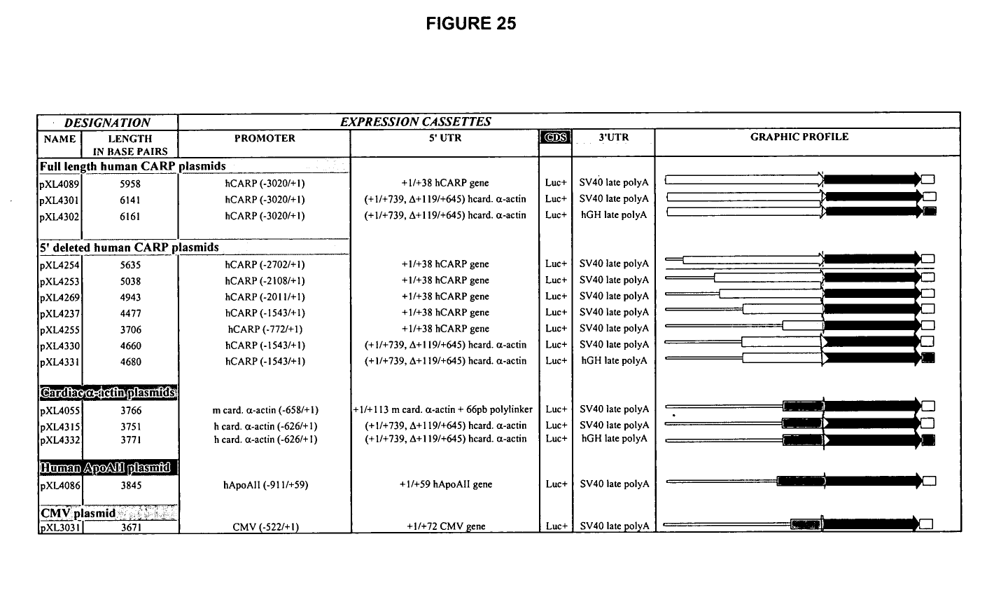

The activity of different tissue-specific promoters in cardiac tissue was assessed. FIG. 25 shows the construction of DNA plasmids with different tissue-specific promoters (e.g., complete or 5′deleted human CARP promoters, mouse cardiac actin promoters, and liver-specific human ApoAII promoter). The plasmids contain an expression cassette for the modified firefly (Photinus pyralis) luciferase reporter gene (luc+). This expression cassette was inserted into an original backbone (pCOR), to create a closed circular plasmid. The late polyadenylation signal from Simian Virus 40 was inserted downstream of the luciferase gene to ensure proper and efficient transcription termination and the polyadenylation of luciferase transcripts. Quality control analyses confirmed the identity, purity, and proper construction of the various plasmids.

The various promoter-carrying plasmids were injected into the left ventricle of SD rats to compare their relative abilities to induce luciferase gene expression in cardiac tissue.

In particular, the plasmid containing the full-length hCARP promoter plasmid, i.e., pXL4089 (−3020/+38, SEQ ID NO: 13), was prepared as a stock solution of 0.886 mg/ml in 150 mM NaCl. Also, 5′ deleted hCARP promoter plasmids were prepared as follows: plasmid pXL4254 (−2702/+38, SEQ ID NO: 3), 0.829 mg/ml in 150 mM NaCl; plasmid pXL4253 (−2108/+38, SEQ ID NO: 4), 1.521 mg/ml in 150 mM NaCl; plasmid pXL4269 (−2011/+38, SEQ ID NO: 5), 1.041 mg/ml in 150 mM NaCl; plasmid pXL4267 (−2702/+38, SEQ ID NO: 3), 1.628 mg/ml in 150 mM NaCl; plasmid pXL4237 (−1543/+38, SEQ ID NO: 6), 1.203 mg/ml in 150 mM NaCl; and plasmid pXL4255 (−772/+38, SEQ ID NO: 7), 0.921 mg/ml in 150 mM NaCl.

Murine cardiac α actin promoters were used as positive controls for cardiac expression of luciferase and were prepared as follows: plasmid pXL4081, 1.019 mg/ml in 150 mM NaCl; and plasmid pXL4055, 1.364 mg/ml in 150 mM NaCl.

The liver-specific promoter (hApoAll) was used as negative control, and was prepared as follows: plasmid pXL4086, 1.456 mg/ml in 150 mM NaCl.

Male OFA Sprague Dawley rats (250 g) were supplied by IFFA CREDO (BP109, L'Arbresle, France). The rats were anesthetized by an intraperitoneal injection of xylazine (6 mg/kg) and ketamine (70 mg/kg). After laparotomia, the various plasmids (25 μg in 50 μl of 150 mM NaCl) were injected through the diaphragm and directly into the left ventricle (at the heart apex). The injection was performed with a 100 μl Hamilton syringe, which was coupled to a catheter (Steriflex ref 167.10 G19V) equipped with a thrust block and a needle (BD 26G*3/8). The surgical procedure was completed by stitching the musculature with Ethicon Vicryl JV294 and the skin with Ethicon Mersutures FR870. The study included 5 independent experiments. In each experiment, the rats were divided into 10 groups (one for each plasmid tested) of 2 or 4 rats.

Animals were euthanathized by an intraperitoneal injection of an overdose of pentobarbital (400 μl) three days after plasmid administration. Each heart was excised, washed 2 times in 150 mM NaCl, and weighed after removal of the auricles. The tissue was homogenized (polytron PT 1200CL) in 10 ml of lysis buffer (5×Luciferase Assay Cell Culture Lysis Reagent; Promega E153A) containing protease inhibitors at 4 μg/ml (Protease Inhibitor Cocktail Tablets; Roche Diagnostics). The homogenate was then centrifuged for 20 min at 4,000 rpm at 4° C.

The luciferase activity (expressed as counts per second (CPS)/heart) present in 10 μl of supernatant was assayed using the Promega Luciferase Assay System and a luminometer. Data shown are the geometric means of replicate samples, and error bars represent the confidence interval at 95%. Due to the variability of luciferase expression in the injected heart, the results were normalized using a log transformation and were subjected to statistical analysis using a Newman-Keuls test on the pooled data from the 5 experiments.

FIG. 29 shows that the murine cardiac actin promoter (pXL4055) was less active (28%) than the full-length CARP promoter in rat heart. In addition, the results indicated that the liver-specific hApoAII promoter (pXL4086) was even less active than the full-length CARP promoter (5%) and was comparable to the background luciferase activity. In contrast, 5′ deletions of the human CARP promoter did not affect its activity compared to the full length CARP promoter 3 days after plasmid injection into the heart.

1. A polynucleotide comprising a fragment of any one of SEQ ID NOs: 3 to 7, or a fragment of a sequence that hybridizes under high stringency conditions with any one of SEQ ID Nos: 3 to 7, wherein said polynucleotide in the absence of inverted terminal repeat sequences from adeno-associated virus specifically induces expression in cardiac cells in vivo of a gene which is operably linked to said polynucleotide.

2. The polynucleotide according to claim 1, wherein said polynucleotide is SEQ ID NO: 3, or a sequence hybridizing under high stringency conditions with SEQ ID NO: 3.

3. The polynucleotide according to claim 1, wherein said polynucleotide is SEQ ID NO: 4, or a sequence hybridizing under high stringency conditions with SEQ ID NO: 4.

4. The polynucleotide according to claim 1, wherein said polynucleotide is SEQ ID NO: 5, or a sequence hybridizing under high stringency conditions with SEQ ID NO: 5.

5. The polynucleotide according to claim 1, wherein said polynucleotide is SEQ ID NO: 6, or a sequence hybridizing under high stringency conditions with SEQ ID NO: 6.

6. The polynucleotide according to claim 1, wherein said polynucleotide is SEQ ID NO: 7, or a sequence hybridizing under high stringency conditions with SEQ ID NO: 7.

7. An expression cassette comprising a sequence encoding a protein or an RNA of therapeutic interest operably linked to the polynucleotide according to claim 1.

8. The expression cassette according to claim 7, further comprising a polynucleotde SEQ ID NO: 9 operably linked to the polynucleotide according to claim 1.

9. The expression cassette according to claim 7, wherein the protein or RNA of therapeutic interest increases a rate of cardiac cell division, reduces or suppresses an immune response, induces angiogenesis, changes muscle contractility, reduces cardiac hypertrophy, reduces cardiac insufficiency, or reduces myocarditis.

10. The expression cassette according to claim 9, wherein the protein or RNA of therapeutic interest is a vascular endothelial growth factor, a fibroblast growth factor, an angiopoietin, or a cytokine.

11. The expression cassette according to claim 9, wherein the protein of therapeutic interest is an immunosuppressive protein.

12. The expression cassette according to claim 11, wherein the immunosuppressive protein is interleukin-10, interleukin-2, or interleukin-8.

13. The expression cassette according to claim 9, wherein the protein of therapeutic interest reduces hypoxia.

14. The expression cassette according to claim 13, wherein the protein that reduces hypoxia is nitric oxide synthetase, superoxide dismutase, or catalase.

15. A vector comprising the polynucleotide according to claim 1.

16. The vector according to claim 15, further comprising an origin of replication which is active in cardiac cells.

17. The vector according to claim 15, which is a plasmid, a cosmid, or any DNA not encapsidated by viral proteins

18. The vector according to claim 15, which is or is derived from an adenovirus, a retrovirus, a herpesvirus, or an adeno-associated virus.

19. A composition comprising a therapeutically-effective amount of the vector according to claim 15 and a pharmaceutically-acceptable carrier.

20. A method for expressing a protein or an RNA of therapeutic interest in cardiac cells in vivo, comprising
preparing a vector according to claim 15, and
introducing said vector into cardiac cells in vivo so that said protein or RNA of therapeutic interest is expressed.
